(19) United States
(12) Patent Application Publication
(10) Pub. No.: US 2023/0005567 A1
(43) Pub. Date: Jan. 5, 2023

(54) GENERATING PROTEIN SEQUENCES USING MACHINE LEARNING TECHNIQUES BASED ON TEMPLATE PROTEIN SEQUENCES

(71) Applicant: Just- Evotec Biologics, Inc., Seattle, WA (US)

(72) Inventors: Jeremy Martin Shaver et al., Lake Forest Park, WA (US); Tileli Amimeur, Seattle, WA (US); Randal Robert Ketchem, Snohomish, WA (US); Alex Taylor, Bellevue, WA (US)

(21) Appl. No.: 17/784,576

(22) PCT Filed: Dec. 11, 2020
(86) PCT No.: PCT/US2020/064579

§ 371 Date: Jun. 10, 2022

Related U.S. Application Data
(60) Provisional application No. 62/947,430, filed on Dec. 12, 2019.

(51) Int. Cl. G16B 20/00; G16B 30/10

(52) U.S. Cl. CPC G16B 20/00; G16B 30/10

(57) ABSTRACT

Systems and techniques are described to generate amino acid sequences of target proteins based on amino acid sequences of template proteins using machine learning techniques. The amino acid sequences of the target proteins can be generated based on data that constrains the modifications that can be made to the amino acid sequences of the template proteins. In illustrative examples, the template proteins can include antibodies produced by a non-human mammal that bind to an antigen and the target proteins can correspond to human antibodies with a region having at least a threshold amount of identity with the binding region of the template antibody. Generative adversarial networks can be used to produce the amino acid sequences of the target proteins.

### BACKGROUND

Proteins are biological molecules that are comprised of one or more chains of amino acids. Proteins can have various functions within an organism. For example, some proteins can be involved in causing a reaction to take place within an organism. In other examples, proteins can transport molecules throughout the organism. In still other examples, proteins can be involved in the replication of genes. Additionally, some proteins can have therapeutic properties and can be used to treat various biological conditions. The structure and function of proteins are based on the arrangement of amino acids that comprise the proteins. The arrangement of amino acids for proteins can be represented by a sequence of letters with each letter corresponding to an amino acid at a certain position of the protein. The arrangement of amino acids for proteins can also be represented by three dimensional structures that not only indicate the amino acids at certain positions of the protein, but also indicate three dimensional features of the proteins, such as an α-helix or a β-sheet.

### BRIEF DESCRIPTION OF THE DRAWINGS

The present disclosure is illustrated by way of example and not limitation in the figures of the accompanying drawings, in which like references indicate similar elements.

FIG. 1 is a diagram illustrating an example framework to generate target protein sequences using machine learning techniques based on template protein sequences, in accordance with some implementations.

FIG. 2 is a diagram illustrating an example framework to utilize transfer learning techniques to generate protein sequences having specified characteristics, in accordance with some implementations.

FIG. 3 is a diagram illustrating an example framework to generate target protein sequences using a generative adversarial network based on a template protein sequence and constraint data related to modifications of positions of the template sequence, in accordance with some implementations.

FIG. 4 is a diagram illustrating an example framework to utilize data indicating an antibody sequence of a first organism having specified functionality to generate data corresponding to additional antibody sequences having the specified functionality for a second, different organism, in accordance with some implementations.

FIG. 5 is a diagram illustrating an example framework to generate target protein sequences using machine learning techniques by combining protein fragment sequences with template protein sequences, in accordance with some implementations.

FIG. 6 is a flow diagram illustrating an example method for producing target protein sequences using template protein sequences and position modification data, in accordance with some implementations.

FIG. 7 is a flow diagram illustrating an example method for producing target protein sequences using a generative adversarial network based on template protein sequences, in accordance with some implementations.

FIG. 8 illustrates a diagrammatic representation of a machine in the form of a computer system within which a set of instructions may be executed for causing the machine to perform any one or more of the methodologies discussed herein, according to an example embodiment.

### DETAILED DESCRIPTION

Proteins can have many beneficial uses within organisms. For example, proteins can be used to treat diseases and other biological conditions that can detrimentally impact the health of humans and other mammals. In various scenarios, proteins can participate in reactions that are beneficial to subjects and that can counteract one or more biological conditions being experienced by the subjects. In some examples, proteins can also bind to molecules within an organism that may be detrimental to the health of a subject. In various situations, the binding of proteins to potentially harmful molecules can result in activation of the immune system of a subject to neutralize the potential effects of the molecules. For these reasons, many individuals and organizations have sought to develop proteins that may have therapeutic benefits.

The development of proteins for use in treating biological conditions can be a time consuming and resource intensive process. Often, candidate proteins for development can be identified as potentially having desired biophysical properties, three-dimensional (3D) structures, and/or behavior within an organism. In order to determine whether the candidate proteins actually have the desired characteristics, the proteins can be physically synthesized and then tested to determine whether the actual characteristics of the synthesized proteins correspond to the desired characteristics. Due to the amount of resources needed to synthesize and test proteins for specified biophysical properties, 3D structures, and/or behaviors, the number of candidate proteins synthesized for therapeutic purposes is limited. In some situations, the number of proteins synthesized for therapeutic purposes can be limited by the loss of resources that takes place when candidate proteins are synthesized and do not have the desired characteristics.

The use of computer-implemented techniques to identify candidate proteins that have particular characteristics has increased. These conventional techniques, however, can be limited in their scope and accuracy. In various situations, conventional computer-implemented techniques to generate protein sequences can be limited by the amount of data available and/or the types of data available that may be needed by those conventional techniques to accurately generate protein sequences with specified characteristics. Additionally, the techniques utilized to produce models that can generate protein sequences with particular characteristics can be complex and the know-how needed to produce models that are accurate and efficient can be complex and difficult to implement. The length of the protein sequences produced by conventional models can also be limited because the accuracy of conventional techniques can decrease as the lengths of the proteins increases and because the computing resources used to generate large numbers of protein sequences, such as hundreds, thousands, up to millions of protein sequences, having a relatively large number of amino acids (e.g., 50-1000) can become prohibitive. Thus, the number of proteins generated by conventional computational techniques is limited.

Further, although proteins produced by one organism or type of organism can have functionality that may be beneficial to a number of organisms, in various scenarios, the same proteins can be rejected by the immune system of another organism or type of organism and obviate the beneficial functionality of the proteins. The techniques and systems described herein can be used to generate amino acid sequences of target molecules based on amino acid sequences of template molecules. The template molecules can exhibit a functionality that can be beneficial for a number of different organisms besides the original host that produced the template molecules. The target molecules can also exhibit the functionality of the template molecules, while minimizing the possibility of rejection by an organism that is different from the original host.

For example, the portions of the amino acid sequence of a template protein that are attributed to a functionality of the template protein within a host organism can be preserved, while additional portions of the amino acid sequence of the template protein can be modified to minimize the possibility of rejection by another organism. To illustrate, a template antibody produced in a mouse can effectively bind to an antigen that is found in both mice and humans. The binding of the template antibody to the antigen can be attributed to one or more binding regions of the template antibody. The techniques and systems described herein can generate data corresponding to a number of amino acid sequences for target antibodies that include the binding regions of the template antibody and that also include additional regions that have been modified from the template antibody that correspond to amino acid sequences included in human antibodies. In this way, the techniques and systems described herein can produce an antibody with a human framework in conjunction with binding regions for a specific antigen, where the binding regions for the antigen may not be present in known human antibodies. Accordingly, biological conditions that may not have been responsive to known human antibodies can be treated using antibodies with amino acid sequences generated from the techniques and systems described herein.

Machine learning techniques can be used to generate the target protein amino acid sequences from the template protein amino acid sequences. In illustrative examples, generative adversarial networks can be used to generate the target protein amino acid sequences. The generative adversarial networks can be trained using target protein amino acid sequences in relation to template protein amino acid sequences and position modification data. The position modification data can indicate, for individual positions of a template protein amino acid sequence, a likelihood that the amino acid can be modified to a different amino acid. In various implementations, the position modification data can correspond to a penalty applied by the generative adversarial network in response to modification of an individual amino acid. For example, a position of a template protein amino acid sequence having a relatively high penalty for being modified can be less likely to be modified by the generative adversarial network, while another position of the template protein amino acid sequence having a relatively low penalty for being modified can be more likely to be modified by the generative adversarial network. In various examples, transfer learning techniques can also be applied to produce target antibodies having one or more biophysical properties.

The position modification data can be based on the location of the amino acids in the template protein sequence. Amino acids located in regions of the template protein associated with a desired functionality can have a relatively high penalty for being modified, while amino acids located in other regions of a template protein can have relatively moderate or relatively low penalties for being modified. In situations where a target protein corresponds to a different organism than a host organism that produces the template protein, the positions of the template protein associated with relatively low penalties for being modified can be the most likely to be changed to correspond to a framework for the organism related to the target protein. Additionally, in scenarios where the target protein is derived from a germline gene that is different from the germline gene of the host that produces the template protein, the positions of the template protein associated with relatively low penalties for being modified can be the most likely to be changed to correspond to a protein produced from the target protein germline gene. As used herein, germline, can correspond to amino acid sequences of proteins that are conserved when cells of the proteins replicate. An amino acid sequence can be conserved from a parent cell to a progeny cell when the amino acid sequence of the progeny cell has at least a threshold amount of identity with respect to the corresponding amino acid sequence in the parent cell. In an illustrative example, a portion of an amino acid sequence of a human antibody that is part of a kappa light chain that is conserved from a parent cell to a progeny cell can be a germline portion of the antibody.

In illustrative examples, an antibody produced in mice can bind to an antigen that is found in both mice and humans. The binding of the antibody to the antigen can be based on amino acids located in the complementarity-determining regions (CDRs) of the antibody. In this scenario, the position modification data can indicate relatively high penalties for changing amino acids located in the CDRs of the template mouse antibody. The position modification data can also indicate lower penalties for the modification of amino acids located in the constant domains and other portions of the variable domains of the template mouse antibody. Thus, the generative adversarial networks described herein can generate target human antibody amino acid sequences that preserve most or all of the residues of the mouse antibody that participate in binding the antigen, while changing constant domains and/or other parts of the variable domains of the heavy chains and/or light chains of the mouse antibody to correspond to the heavy chains and light chains of human antibodies. The generative adversarial networks described herein can also be trained using human antibodies in order to determine the characteristics of human antibodies and identify changes to the template mouse antibody that can be made to produce a humanized target antibody for the antigen.

By implementing the techniques and systems described herein, target protein amino acid sequences can be generated based on one or more template proteins amino acid sequences that can preserve at least some functionality of the template proteins, while utilizing a different supporting framework for the portions of the template proteins that are attributed to the functionality The computational and machine learning techniques described herein can efficiently generate target protein amino acid sequences, while minimizing the probability that the target proteins will lose functionality of the template proteins. The techniques and systems described herein can also minimize the probability that the target proteins will be rejected by an organism that is different from the host organism that produced the template proteins. For example, the use of position modification data can decrease the amount of computing resources utilized in generating target protein sequences by constraining the number of changes that can be made by a computational model to the template protein sequences, while allowing for flexibility in portions of the template sequences that are less constrained to coincide with features of target proteins related to the new host organism. In various examples, the techniques and systems described herein can analyze thousands up to millions of amino acid sequences of proteins to accurately generate amino acid sequences of new proteins that both preserve the functionality of template proteins while also minimizing the probability of the new proteins being rejected by a new host organism.

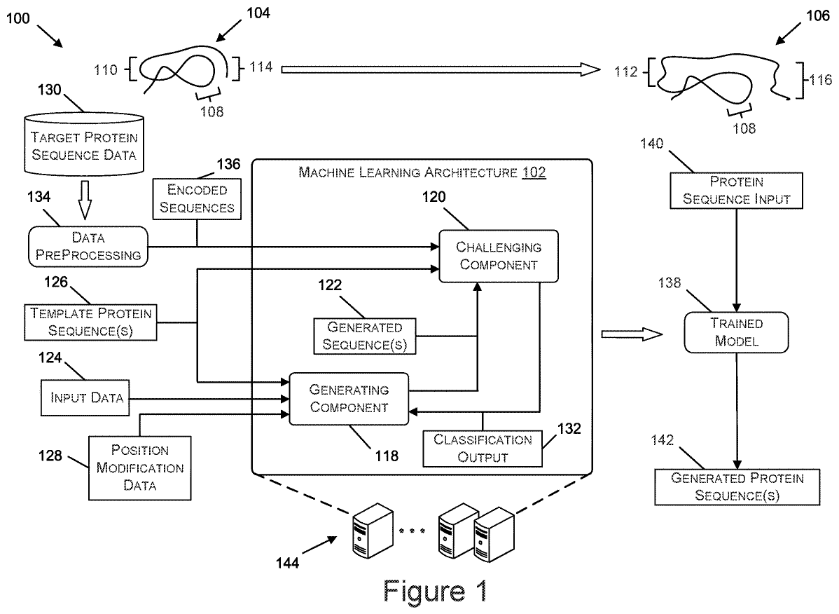

FIG. 1 is a diagram illustrating an example framework 100 to generate target protein sequences using machine learning techniques based on template protein sequences, in accordance with some implementations. For example, a machine learning architecture 102 can obtain an amino acid sequence of a template protein 104 and generate an amino acid sequence of a target protein 106. The template protein 104 can include a region 108 that has a functionality and the machine learning architecture 102 can generate the target protein 106 such that the target protein 106 also includes the region 108. In various implementations, target proteins include a region having at least a threshold amount of identity with the region 108. In this way, the target protein 106 can retain the functionality of the template protein 104. To illustrate, the machine learning architecture 102 can generate the target protein 106 to maximize a probability that the target protein 106 retains the functionality attributed to the region 108 by preserving at least a threshold amount of the region 108 and/or preserving amino acids at various locations of the region 108.

In illustrative examples, an amount of sequence identity between the region 108 of the template protein 104 and a portion of a target protein 106 can indicate that at least a portion of the region 108 of the template protein 104 and the portion of the target protein 106 have the same nucleotide at a number of positions. An amount of identity between at least a portion of the region 108 of the template protein 104 and a portion of the target protein 106 can be determined using a Basic Local Alignment Search Tool (BLAST).

Additional portions of the target protein 106 can have different amino acid sequences in relation to portions of the template protein 104. The regions of the target protein 106 that have different amino acid sequences in relation to portions of the template protein 104 can also have one or more different secondary structures in relation to the secondary structures of the template protein 104. The differences between the amino acid sequences of regions of the template protein 104 and the regions of the target protein 106 can also result in different tertiary structures for the template protein 104 and the target protein 106. In the illustrative example of FIG. 1, the template protein 104 can include a region 110 that has a different amino acid sequence from a region 112 of the target protein 106. Further, the template protein 104 can include a region 114 that has a different amino acid sequence from a region 116 of the target protein 106.

The machine learning architecture 102 can modify regions of the template protein 104 to produce the amino acid sequence of the target protein 106 such that portions of the amino acid sequence of the target protein 106 correspond to proteins produced by a different organism than the organism that produced the template protein 104. For example, the template protein 104 can be produced by one mammal and the target protein 106 can be produced by a different mammal. To illustrate, the template protein 104 can be produced by mice and the target protein 106 can correspond to proteins produced by humans. In additional examples, the template protein 104 can correspond to a protein produced in relation to a first germline gene and the target protein 106 can correspond to a protein produced in relation to a second germline gene. In situations where the template protein 104 and the target protein 106 are antibodies, the template protein 104 can have an amino acid sequence that corresponds to a first antibody isotype (e.g., immunoglobin E (IgE)) and the target protein 106 can have an amino acid sequence that corresponds to a second antibody isotype (e.g., IgG).

The machine learning architecture 102 can include a generating component 118 and a challenging component 120. The generating component 118 can implement one or more models to generate amino acid sequences based on input provided to the generating component 118. In various implementations, the one or more models implemented by the generating component 118 can include one or more functions. The challenging component 120 can generate output indicating whether the amino acid sequences produced by the generating component 118 satisfy various characteristics. The output produced by the challenging component 120 can be provided to the generating component 118 and the one or more models implemented by the generating component 118 can be modified based on the feedback provided by the challenging component 120. The challenging component 120 can compare the amino acid sequences produced by the generating component 118 with amino acid sequences of a library of target proteins and generate an output indicating an amount of correspondence between the amino acid sequences produced by the generating component 118 and the amino acid sequences of target proteins provided to the challenging component 120.

In various implementations, the machine learning architecture 102 can implement one or more neural network technologies. For example, the machine learning architecture 102 can implement one or more recurrent neural networks. Additionally, the machine learning architecture 102 can implement one or more convolution neural networks. In certain implementations, the machine learning architecture 102 can implement a combination of recurrent neural networks and convolutional neural networks. In examples, the machine learning architecture 102 can include a generative adversarial network (GAN). In these situations, the generating component 118 can include a generator and the challenging component 120 can include a discriminator. In additional implementations, the machine learning architecture 102 can include a conditional generative adversarial network (cGAN).

In the illustrative example of FIG. 1, data can be provided to the generating component 118 and the generating component 118 can utilize the data and one or more models to produce generated sequences 122. The generated sequences 122 can include amino acid sequences that are represented by a series of letters with each letter indicating an amino acid located at a respective position of a protein. The data provided to the generating component 118 to produce the generated sequences 122 can include input data 124. The input data 124 can include noise that is produced by a random number generator or noise produced by a pseudo-random number generator. In addition, the data provided to the generating component 118 to produce the generated sequences 122 can include one or more template protein sequences 126. A template protein sequence 126 can include an amino acid sequence of a protein that has one or more characteristics that are desirable to include in proteins that are different from a template protein, such as the template protein 104. In illustrative examples, the template protein sequences 126 can correspond to antibodies that bind to a specified antigen. In additional examples, the template protein sequences 126 can correspond to proteins that transport one or more metals through a body of a mammal.

Additionally, position modification data 128 can be provided to the generating component 118 to be used by the generating component 118 to produce the generated sequences 122. The position modification data 128 can indicate one or more criteria related to the modification of amino acids of the one or more template protein sequences 126. For example, the position modification data 128 can indicate one or more criteria corresponding to the modification of individual amino acids of the one or more template protein sequences 126. To illustrate, the position modification data 128 can indicate respective probabilities that amino acids at individual positions of a template protein sequence 126 can be modified. In additional implementations, the position modification data 128 can indicate a penalty associated with the modification of amino acids at individual positions of a template protein sequence 126. The position modification data 128 can include values or functions corresponding to the respective amino acids located at individual positions of a template protein sequence 126.

In illustrative examples, the position modification data 128 can include criteria that reduce the probability of amino acids being modified at positions of a template protein that correspond to functionality of the template protein that is to be preserved in a target protein. For example, a penalty associated with modifying an amino acid located in a region that is attributed to functionality of a template protein can be relatively high. Additionally, the position modification data 128 can include criteria for amino acids outside of one or more regions that are attributed to functionality of a template protein that indicate increased or neutral probabilities for modification of those amino acids. To illustrate, a penalty associated with modifying an amino acid located at a position outside of a region attributed to particular functionality of a protein can be relatively low or neutral. Further, the position modification data 128 can indicate probabilities of changing amino acids at positions of a template protein to different types of amino acids. In illustrative examples, an amino acid located at a position of a template protein can have a first penalty for being changed to a first type of amino acid and a second, different penalty for being changed to a second type of amino acid. That is, in various implementations, a hydrophobic amino acid of a template protein can have a first penalty for being changed to another hydrophobic amino acid and a second, different penalty for being changed to a positively charged amino acid.

In one or more examples, the position modification data 128 can be determined, at least in part, based on input obtained via a computing device. For example, a user interface can be generated that includes one or more user interface elements to capture at least a portion of the position modification data 128. In addition, a data file can be obtained over a communication interface that includes at least a portion of the position modification data 128. Further, the position modification data 128 can be computed by analyzing a number of amino acid sequences to determine numbers of occurrences of different amino acids at one or more positions of the proteins. Occurrences of amino acids at positions of proteins, including template proteins and target proteins, can be used to determine probabilities of modifications of amino acids that are indicated in the position modification data 128. In various examples, biophysical properties and/or structural properties of proteins can be analyzed in conjunction with the placement of amino acids at one or more positions of template proteins and target proteins to determine probabilities included in the position modification data 128 for modifying amino acids at one or more positions of template proteins to generate target proteins.

The generated sequence(s) 122 can be compared by the challenging component 120 against sequences of proteins included in target protein sequence data 130. The target protein sequence data 130 can be training data for the machine learning architecture 102. The target protein sequence data 130 can be encoded according to a schema. A schema applied to amino acid sequences included in the target protein sequence data 130 can be based on a classification of the amino acid sequences. For example, an antibody can be stored according to a first classification, a signaling protein can be stored according to a second classification, and a transport protein can be stored according to a third classification.

The target protein sequence data 130 can include sequences of proteins obtained from one or more data sources that store amino acid sequences of proteins. The one or more data sources can include one or more websites that are searched and information corresponding to amino acid sequences of target proteins can be extracted from the one or more websites. Additionally, the one or more data sources can include electronic versions of research documents from which amino acid sequences of target proteins can be extracted.

In illustrative examples, the target protein sequence data 130 can include amino acid sequences of proteins that are produced by an organism that is different from an organism that produces the template protein sequences 126. For example, the target protein sequence data 130 can include amino acid sequences of human proteins and the one or more template protein sequences 126 can correspond to one or more proteins produced by mice or chickens. In additional examples, the target protein sequence data 130 can include amino acid sequences of horse proteins and the one or more template protein sequences 126 can correspond to one or more proteins produced by humans. In various examples, the amino acid sequences included in the target protein sequence data 130 can have one or more characteristics and/or functions. To illustrate, the amino acid sequences included in the target protein sequence data 130 can correspond to human enzymes used in the metabolism of various foods consumed by humans. In further examples, the amino acid sequences included in the target protein sequence data 130 can correspond to human antibodies.

The template protein sequences 126, the position modification data 128, the target protein sequence data 130, or combinations thereof, can be stored in one or more data stores that are accessible to the machine learning architecture 102. The one or more data stores can be connected to the machine learning architecture 102 via a wireless network, a wired network, or combinations thereof. The template protein sequences 126, the position modification data 128, the target protein sequence data 130, or combinations thereof, can be obtained by the machine learning architecture 102 based on requests sent to the data stores to retrieve one or more portions of at least one of the template protein sequences 126, the position modification data 128, or the target protein sequence data 130.

The challenging component 120 can generate output indicating whether the amino acid sequences produced by the generating component 118 satisfy various characteristics. In one or more implementations, the challenging component 120 can be a discriminator. In additional situations, such as when the machine learning architecture 102 includes a Wasserstein GAN, the challenging component 120 can include a critic.

In illustrative examples, based on similarities and differences between the generated sequence(s) 122 and additional sequences provided to the challenging component 120, such as amino acid sequences included in the target protein sequence data 130, the challenging component 120 can generate the classification output 132 to indicate an amount of similarity or an amount of difference between the generated sequence(s) 122 and sequences provided to the challenging component 120 that are included in the target protein sequence data 130. Additionally, the classification output 132 can indicate an amount of similarity or an amount of difference between the generated sequence(s) 122 and the template protein sequences 126.

In one or more examples, the challenging component 120 can label the generated sequence(s) 122 as zero and the encoded sequences obtained from the target protein sequence data 130 as 1. In these situations, the classification output 132 can include a first number from 0 to 1 with respect to one or more amino acid sequences included in the target protein sequence data 130. Additionally, the challenging component 120 can label the generated sequences 122 as zero and the template protein sequences 126 as 1. Accordingly, the challenging component 120 can generate another number from 0 to 1 with respect to the template protein sequences 126.

In additional examples, the challenging component 120 can implement a distance function that produces an output that indicates an amount of distance between the generated sequence(s) 120 and the proteins included in the target protein sequence data 130. Further, the challenging component 120 can implement a distance function that produces an output that indicates an amount of distance between the generated sequence(s) 122 and the template protein sequence(s) 126. In implementations where the challenging component 120 implements a distance function, the classification output 132 can include a number from −∞ to ∞ indicating a distance between the generated sequence(s) 122 and one or more sequences included in the target protein sequence data 130. The challenging component 120 can also implement a distance function and generate a classification output 132 including an additional number from −∞ to ∞ indicating a distance between the generated sequence(s) 122 and the template protein sequences 126.

The amino acid sequences included in the target protein sequence data 130 can be subject to data preprocessing 134 before being provided to the challenging component 120. For example, the target protein sequence data 130 can be arranged according to a classification system before being provided to the challenging component 120. The data preprocessing 134 can include pairing amino acids included in the target proteins of the target protein sequence data 130 with numerical values that can represent structure-based positions within the proteins. The numerical values can include a sequence of numbers having a starting point and an ending point. In an illustrative example, a T can be paired with the number 43 indicating that a Threonine molecule is located at a structure-based position 43 of a specified protein domain type. In illustrative examples, structure-based numbering can be applied to any general protein type, such as fibronectin type III (FNIII) proteins, avimers, antibodies, VHH domains, kinases, zinc fingers, T-cell receptors, and the like.

In various implementations, the classification system implemented by the data preprocessing 134 can include a numbering system that encodes structural position for amino acids located at respective positions of proteins. In this way, proteins having different numbers of amino acids can be aligned according to structural features. For example, the classification system can designate that portions of proteins having particular functions and/or characteristics can have a specified number of positions. In various situations, not all of the positions included in the classification system may be associated with an amino acid because the number of amino acids in a particular region of a protein may vary between proteins. In additional examples, the structure of a protein can be reflected in the classification system. To illustrate, positions of the classification system that are not associated with a respective amino acid can indicate various structural features of a protein, such as a turn or a loop. In an illustrative example, a classification system for antibodies can indicate that heavy chain regions, light chain regions, and hinge regions have a specified number of positions assigned to them and the amino acids of the antibodies can be assigned to the positions according to the classification system. In one or more implementations, the data preprocessing 134 can use Antibody Structural Numbering (ASN) to classify individual amino acids located at respective positions of an antibody.

The data used to train the machine learning architecture 102 can impact the amino acid sequences produced by the generating component 118. For example, in situations where human antibodies are included in the protein sequence data 130 provided to the challenging component 120, the amino acid sequences generated by the generating component 118 can correspond to human antibody amino acid sequences. In another example, in scenarios where the amino acid sequences included in the target protein sequence data 130 provided to the challenging component 120 correspond to proteins produced from a germline gene, the amino acid sequences produced by the generating component 118 can correspond to proteins produced from the germline gene. Further, when the amino acid sequences included in the target protein sequence data 130 provided to the challenging component 120 correspond to antibodies of a specified isotype, the amino acid sequences produced by the generating component 118 can correspond to antibodies of the specified isotype.

The output produced by the data preprocessing 134 can include encoded sequences 136. The encoded sequences 136 can include a matrix indicating amino acids associated with various positions of a protein. In examples, the encoded sequences 136 can include a matrix having columns corresponding to different amino acids and rows that correspond to structure-based positions of proteins. For each element in the matrix, a 0 can be used to indicate the absence of an amino acid at the corresponding position and a 1 can be used to indicate the presence of an amino acid at the corresponding position. The matrix can also include an additional column that represents a gap in an amino acid sequence where there is no amino acid at a particular position of the amino acid sequence. Thus, in situations where a position represents a gap in an amino acid sequence, a 1 can be placed in the gap column with respect to the row associated with the position where an amino acid is absent. The generated sequence(s) 122 can also be represented using a vector according to a same or similar number scheme as used for the encoded sequences 136. In some illustrative examples, the encoded sequences 136 and the generated sequence(s) 122 can be encoded using a method that may be referred to as a one-hot encoding method.

After the machine learning architecture 102 has undergone a training process, a trained model 138 can be generated that can produce sequences of proteins. The trained model 138 can include the generating component 118 after a training process has been performed using the protein sequence data 130. In illustrative examples, the trained model 138 include a number of weights and/or a number of parameters of a convolution neural network. The training process for the machine learning architecture 102 can be complete after the function(s) implemented by the generating component 118 and the function(s) implemented by the challenging component 120 converge. The convergence of a function can be based on the movement of values of model parameters toward particular values as protein sequences are generated by the generating component 118 and feedback is obtained from the challenging component 120. In various implementations, the training of the machine learning architecture 102 can be complete when the protein sequences produced by the generating component 118 have particular characteristics. For example, the amino acid sequences generated by the generating component 118 can be analyzed by a software tool that determines at least one of biophysical properties of the amino acid sequences, structural features of the amino acid sequences, or adherence to amino acid sequences corresponding to one or more protein germlines The machine learning architecture 102 can produce the trained model 138 in situations where the amino acid sequences produced by the generating component 118 are determined by the software tool to have one or more specified characteristics. In various examples, a software tool used to evaluate the amino acid sequences produced by the generating component 118 can determine that the trained model 138 produces amino acid sequences that have preserved functionality of a template protein.

Protein sequence input 140 can be provided to the trained model 138, and the trained model 138 can produce generated protein sequences 142. The protein sequence input 140 can include one or more template protein sequences, additional position constraint data, and an input vector that can include a random or pseudo-random series of numbers. In an illustrative example, the protein sequence input 140 can include one or more template protein sequences 126. The generated protein sequences 142 produced by the trained model 138 can be represented as a matrix structure that is the same as or similar to the matrix structure used to represent the encoded sequences 136 and/or the generated sequence(s) 122. In various implementations, the matrices produced by the trained model 138 that comprise the generated protein sequences 142 can be decoded to produce a string of amino acids that correspond to the sequence of a target protein. In illustrative examples, the protein sequence input 140 can include the amino acid sequence of the template protein 104 and position modification data indicating a relatively high probability that the amino acids located in the region 108 are to be preserved in order to preserve the functionality of the region 108. The trained model 138 can then use the protein sequence input 140 to generate a number of amino acid sequences of target proteins, such as an amino acid sequence of the target protein 106. In various examples, the trained model 138 can use the protein sequence input 140 to produce hundreds, up to thousands, and up to millions of protein sequences similar to the target protein 106 that correspond to the template protein 104.

Although not shown in the illustrative example of FIG. 1, additional processing can be performed with respect to the generated protein sequences 142. For example, the generated protein sequences 142 can be evaluated to determine whether the generated protein sequences 142 have a specified set of characteristics. To illustrate, one or more metrics can be determined with respect to the target protein sequence(s) 142. For example, metrics that can be determined with respect to the generated protein sequences 142 can be related to characteristics of the generated protein sequences 142, such as a number of negatively charged amino acids, a number of positively charged amino acids, a number of amino acids interacting to form one or more polar regions, amino acids interacting to form one or more hydrophobic regions, one or more combinations thereof, and the like.

The generated protein sequences 142 produced by the trained model 138 can correspond to various types of proteins. For example, the generated protein sequences 142 can correspond to proteins that function as T-cell receptors. In additional examples, the generated protein sequences 142 can correspond to proteins that function as catalysts to cause biochemical reactions within an organism to take place. The generated protein sequences 142 can also correspond to one or more types of antibodies. To illustrate, the generated protein sequences 142 can correspond to one or more antibody subtypes, such as immunoglobin A (IgA), immunoglobin D (IgD), immunoglobin E (IgE), immunoglobin G (IgG), or immunoglobin M (IgM). Further, the generated protein sequences 142 can correspond to additional proteins that bind antigens. In examples, the generated protein sequences 142 can correspond to affibodies, affilins, affimers, affitins, alphabodies, anticalins, avimers, monobodies, designed ankyrin repeat proteins (DARPins), nanoCLAMP (clostridal antibody mimetic proteins), antibody fragments, or combinations thereof. In still other examples, the generated protein sequences 142 can correspond to amino acid sequences that participate in protein-to-protein interactions, such as proteins that have regions that bind to antigens or regions that bind to other molecules.

In some implementations, the generated protein sequences 142 can be subject to sequence filtering. The sequence filtering can parse the generated protein sequences 142 to identify one or more of the generated protein sequences 142 that correspond to one or more characteristics. For example, the generated protein sequences 142 can be analyzed to identify amino acid sequences that have specified amino acids at particular positions. One or more of the generated protein sequences 142 can also be filtered to identify amino acid sequences having one or more particular strings or regions of amino acids. In various implementations, the generated protein sequences 142 can be filtered to identify amino acid sequences that are associated with a set of biophysical properties based at least partly on similarities between at least one of the generated protein sequences 142 and amino acid sequences of additional proteins having the set of biophysical properties.

The machine learning architecture 102 can be implemented by one or more computing devices 144. The one or more computing devices 144 can include one or more server computing devices, one or more desktop computing devices, one or more laptop computing devices, one or more tablet computing devices, one or more mobile computing devices, or combinations thereof. In certain implementations, at least a portion of the one or more computing devices 144 can be implemented in a distributed computing environment. For example, at least a portion of the one or more computing devices 144 can be implemented in a cloud computing architecture. Additionally, although the illustrative example of FIG. 1 shows an implementation of the machine learning architecture 102 that includes a generative adversarial network with a single generating component and a single challenging component, in additional implementations, the machine learning architecture 102 can include multiple generative adversarial networks. Further, each generative adversarial network implemented by the machine learning architecture 102 can include one or more generating components and one or more challenging components.

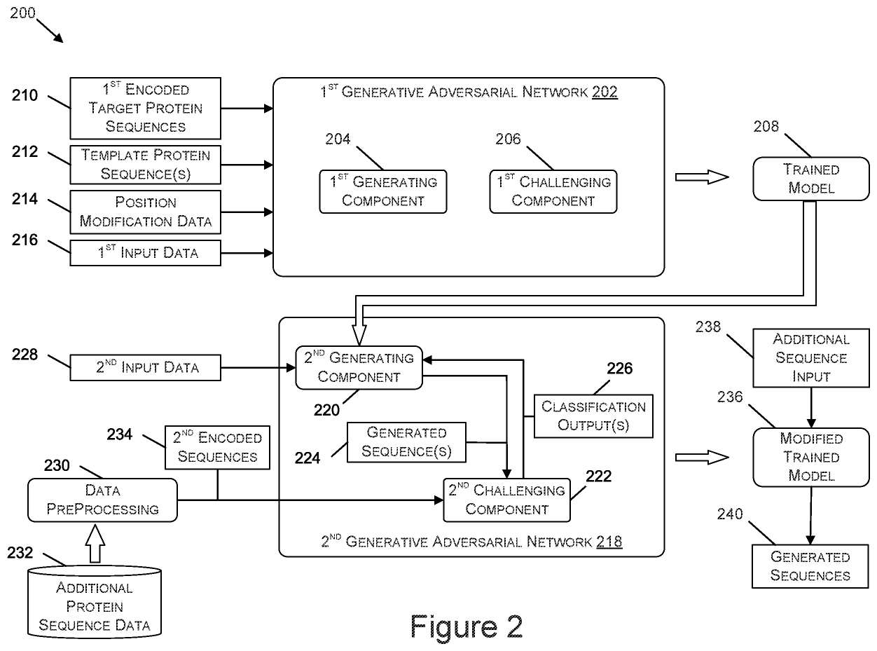

FIG. 2 is a diagram illustrating an example framework 200 to utilize transfer learning techniques to generate protein sequences having specified characteristics, in accordance with some implementations. The framework 200 can include a first generative adversarial network 202. The first generative adversarial network 202 can include a first generating component 204 and a first challenging component 206. In various implementations, the first generating component 204 can be a generator and the first challenging component 206 can be a discriminator. The first generating component 204 can implement one or more models to generate amino acid sequences based on input provided to the first generating component 204. The first challenging component 206 can generate output indicating that the amino acid sequences produced by the generating component 204 satisfy one or more characteristics or output indicating that the amino acid sequences produced by the generating component 204 do not satisfy the one or more characteristics. The output produced by the first challenging component 206 can be provided to the generating component 204 and one or more models implemented by the first generating component 204 can be modified based on the feedback provided by the first challenging component 206. In various implementations, the first challenging component 206 can compare the amino acid sequences produced by the first generating component 204 with amino acid sequences of target proteins and generate an output indicating an amount of correspondence between the amino acid sequences produced by the first generating component 204 and the amino acid sequences of target proteins provided to the first challenging component 206.

The first generative adversarial network 202 can be trained in a same or similar manner described with respect to the machine learning architecture 102 of FIG. 1. For example, first encoded sequences 210 and one or more template protein sequences 212 can be fed into the first challenging component 206 and compared against output produced by the first generating component 204. The output produced by the first generating component 204 can be based on the one or more template protein sequences 212, position modification data 214, and first input data 216. The one or more template protein sequences 212 can include amino acid sequences of proteins that include one or more characteristics that are to be preserved. The position modification data 214 can indicate constraints related to the modification of amino acids at various positions of the one or more template protein sequences 214. The first input data 216 can include data generated by a random number generator or a pseudo-random number generator. The trained model 208 can be produced in response to one or more functions implemented by at least one of the first generating component 204 or the first challenging component 206 satisfying one or more criteria, such as one or more convergence criteria or one or more optimization criteria.

The first encoded target protein sequences 210 can be encoded according to a classification scheme. In addition, the first encoded target protein sequences 210 can include amino acid sequences of target proteins, where the target proteins include a scaffolding or foundational structure that can support one or more functional regions. For example, in situations where the first encoded target protein sequences 210 are human antibodies, the first encoded target protein sequences 210 can have constant regions of light chains and/or heavy chains that are representative of a particular type or class of antibody. To illustrate, the first encoded target protein sequences 210 can include antibodies that have constant regions of heavy chains that correspond to IgA antibodies.

The trained model 208 can generate amino acid sequences of proteins that have at least a portion of the functionality of the one or more template proteins in addition to the underlying structure or scaffold structure of the target proteins. In implementations, the trained model 208 can generate amino acid sequences of human antibodies that bind to an antigen with a CDR that corresponds to a CDR originally found in a mouse antibody. In additional examples, the trained model 208 can generate amino acid sequences of proteins produced from a first germline gene based on input of one or more amino acid sequences of proteins produced from a second, different germline gene.

In additional implementations, the trained model 208 can be generated without using at least one of the template protein sequences 212 or the position modification data 214. For example, the trained model 208 can be generated using the first encoded target protein sequences 210 and the first input data 216. In various implementations, the trained model 208 can be generated using training data for the first generative adversarial network 202 such that the first encoded target protein sequences 210 include amino acid sequences corresponding to one or more germline genes.

In various examples, the amino acid sequences generated by the trained model 208 can be refined further. To illustrate, the trained model 208 can be modified by being subjected to another training process using a different set of training data than the initial training process. For example, the data used for additional training of the trained model 208 can include a subset of the data used to initially produce the trained model 208. In additional examples, the data used for additional training of the trained model 208 can include a different set of data than the data used to initially produce the trained model 208. In illustrative examples, the trained model 208 can produce amino acid sequences of human antibodies with CDR regions of a mouse antibody that binds to an antigen and the trained model 208 can be further refined to generate amino acid sequences of human antibodies with CDR regions originally found in the chicken antibody that have a higher probability of having at least a threshold level of expression in an environment having a specified pH range. Continuing with this example, the trained model 208 can be refined through additional training using a dataset of human antibodies that have a relatively high level of expression in the specified pH range. In the illustrative example of FIG. 2, the refinement of the trained model 208 can be represented by training a second generative adversarial network 218 that includes the training model 208 as the second generating component 220. In various implementations, the second generating component 220 can include the trained model 208 after one or more modifications have been made to the trained model 208. For example, modifications can be made to the trained model 208 in relation to the architecture of the trained model 208, such as the addition of one or more hidden layers or changes to one or more network filters. The second generative adversarial network 218 can also include a second challenging component 222. The second challenging component 222 can include a discriminator.

Second input data 228 can be provided to the second generating component 220 and the second generating component 220 can produce one or more generated sequences 224. The second input data 228 can include a random or pseudo-random sequence of numbers that the second generating component 220 uses to produce the generated sequences 224. The second challenging component 222 can generate second classification output 226 indicating that the amino acid sequences produced by the second generating component 220 satisfy various characteristics or that the amino acid sequences produced by the second generating component 220 do not satisfy various characteristics. In illustrative examples, the second challenging component 222 can generate the classification output 226 based on similarities and differences between one or more generated sequences 224 and amino acid sequences provided to the second challenging component 222. The classification output 226 can indicate an amount of similarity or an amount of difference between the generated sequences 224 and comparison sequences provided to the second challenging component 222.

The amino acid sequences provided to the second challenging component 222 can be included in additional protein sequence data 230. The additional protein sequence data 230 can include amino acid sequences of proteins that have one or more specified characteristics. For example, the additional protein sequence data 230 can include amino acid sequences of proteins having a threshold level of expression in humans. In additional examples, the additional protein sequence data 230 can include amino acid sequences of proteins having one or more biophysical properties and/or one or more structural properties. To illustrate, the proteins included in the additional protein sequence data can have negatively charged regions, hydrophobic regions, a relatively low probability of aggregation, a specified percentage of high molecular weight (HMW), melting temperature, one or more combinations thereof, and the like. In various examples, the additional protein sequence data 230 can include a subset of the protein sequence data used to produce the trained model 208. By providing amino acid sequences to the second challenging component 222 that have one or more specified characteristics, the second generating component 220 can be trained to produce amino acid sequences have at least a threshold probability of having the one or more of the specified characteristics.

Additionally, in many situations where it is desired to produce amino acid sequences of proteins having specific characteristics, the number of sequences available to train a generative adversarial network is limited. In these situations, the accuracy, efficiency, and/or effectiveness of the generative adversarial network to produce amino acid sequences of proteins having the specified characteristics may be unsatisfactory. Thus, without a sufficient number of amino acid sequences available to train a generative adversarial network, the amino acid sequences produced by the generative adversarial network may not have the desired characteristics. By implementing the techniques and systems described with respect to FIG. 2, a first generative adversarial network 202 can perform part of the process of determining amino acid sequences that correspond to proteins or that correspond to a broader class of proteins using a first dataset and the second generative adversarial network 218 can perform additional training to generate amino acid sequences of proteins having more specific characteristics are accurately and efficiently using a second, different dataset. The second dataset can include a subset of the initial training dataset or can include amino acid sequences of proteins having the desired characteristics.

Before being provided to the second challenging component 222, the amino acid sequences included in the additional protein sequence data 230 can be subject to data preprocessing 232. For example, the additional protein sequence data 230 can be arranged according to a classification system before being provided to the second challenging component 222. The data preprocessing 232 can include pairing amino acids included in the amino acid sequences of proteins included in the additional protein sequence data 230 with numerical values that can represent structure-based positions within the proteins. The numerical values can include a sequence of numbers having a starting point and an ending point. The second encoded sequences 234 can include a matrix indicating amino acids associated with various positions of a protein. In various examples, the second encoded sequences 234 can include a matrix having columns corresponding to different amino acids and rows that correspond to structure-based positions of proteins. For each element in the matrix, a 0 can be used to indicate the absence of an amino acid at the corresponding position and a 1 can be used to indicate the presence of an amino acid at the corresponding position. The matrix can also include an additional column that represents a gap in an amino acid sequence where there is no amino acid at a particular position of the amino acid sequence. Thus, in situations where a position represents a gap in an amino acid sequence, a 1 can be placed in the gap column with respect to the row associated with the position where an amino acid is absent. The generated sequence(s) 224 can also be represented using a vector according to a same or similar number scheme as used for the second encoded sequences 234. In some illustrative examples, the second encoded sequences 234 and the second generated sequence(s) 224 can be encoded using a method that may be referred to as a one-hot encoding method. In illustrative examples, the classification system used in the data preprocessing 232 can be the same as or similar to the classification system used in the preprocessing 134 described with respect to FIG. 1. The data preprocessing 232 can produce second encoded sequences 234 that are provided to the second challenging component 222.

The second challenging component 222 can generate output indicating whether the amino acid sequences produced by the second generating component 220 satisfy various characteristics. In various implementations, the second challenging component 222 can be a discriminator. In additional situations, such as when the second generative adversarial network 218 includes a Wasserstein GAN, the second challenging component 222 can include a critic.

In illustrative examples, based on similarities and differences between the generated sequence(s) 224 and additional sequences provided to the second challenging component 222, such as amino acid sequences included in the additional protein sequence data 232, the second challenging component 222 can generate the classification output 226 to indicate an amount of similarity or an amount of difference between the generated sequence(s) 224 and sequences provided to the second challenging component 222 that are included in the additional protein sequence data 232. Additionally, the classification output 226 can indicate an amount of similarity or an amount of difference between the generated sequence(s) 224 and the amino acid sequences included in the additional protein sequence data 232. In additional examples, the second challenging component 222 can implement a distance function that produces an output that indicates an amount of distance between the generated sequence(s) 222 and the proteins included in the additional protein sequence data 232. In implementations where the second challenging component 222 implements a distance function, the classification output 226 can include a number from −∞ to ∞ indicating a distance between the generated sequence(s) 224 and one or more amino acid sequences included in the additional protein sequence data 232.

After the second generative adversarial network 218 has undergone a training process, a modified trained model 236 can be generated that can produce sequences of proteins. The modified trained model 236 can represent the trained model 208 after being trained using the additional protein sequence data 230. In examples, the training process for the second generative adversarial network 218 can be complete after the function(s) implemented by the second generating component 220 and the second challenging component 222 converge. The convergence of a function can be based on the movement of values of model parameters toward particular values as protein sequences are generated by the second generating component 220 and feedback is obtained from the second challenging component 222. The training of the second generative adversarial network 218 can be complete when the protein sequences generated by the second generating component 220 have particular characteristics.

Additional sequence input 238 can be provided to the modified trained model 236, and the modified trained model 236 can produce generated sequences 240. The additional sequence input 238 can include a random or pseudo-random series of numbers and the generated sequences 240 can include amino acid sequences that can be sequences of proteins. In additional implementations, the generated sequences 240 can be evaluated to determine whether the generated sequences 240 have a specified set of characteristics. The evaluation of the generated sequences 240 can produce metrics that indicate characteristics of the generated sequences 240, such as biophysical properties of a protein, biophysical properties of a region of a protein, and/or the presence or absence of amino acids located at specified positions. Additionally, the metrics can indicate an amount of correspondence between the characteristics of the generated sequences 240 and a specified set of characteristics. In some examples, the metrics can indicate a number of positions of a generated sequence 240 that vary from a sequence produced by a germline gene of a protein. Further, an evaluation of the generated sequences 240 can determine the presence or absence of structural features of proteins that correspond to the generated sequences 240.

While the illustrative example of FIG. 2 illustrates the training of a model using multiple training sets in a framework that includes two generative adversarial networks. in additional implementations, the training of a model using multiple training datasets can also be represented using a single generative adversarial network. Further, while the illustrative example of FIG. 2 illustrates the training of a model using generative adversarial networks with two training datasets, in various implementations, more than two datasets can be used to train models using one or more generative adversarial networks according to implementations described herein. For example, the first generating component 204 of the first generative adversarial network 202 can be produced using a previously trained generative adversarial network. To illustrate, the first generating component 204 can be produced using a training data set of amino acid sequences of antibodies and the trained model 208 can be produced using transfer learning techniques with a training data set of amino acid sequences of antibodies that have one or more groups of positions that correspond to a germline gene. The trained model 208 can then be further trained to produce the modified trained model 236 that can generate amino acid sequences of human antibodies.

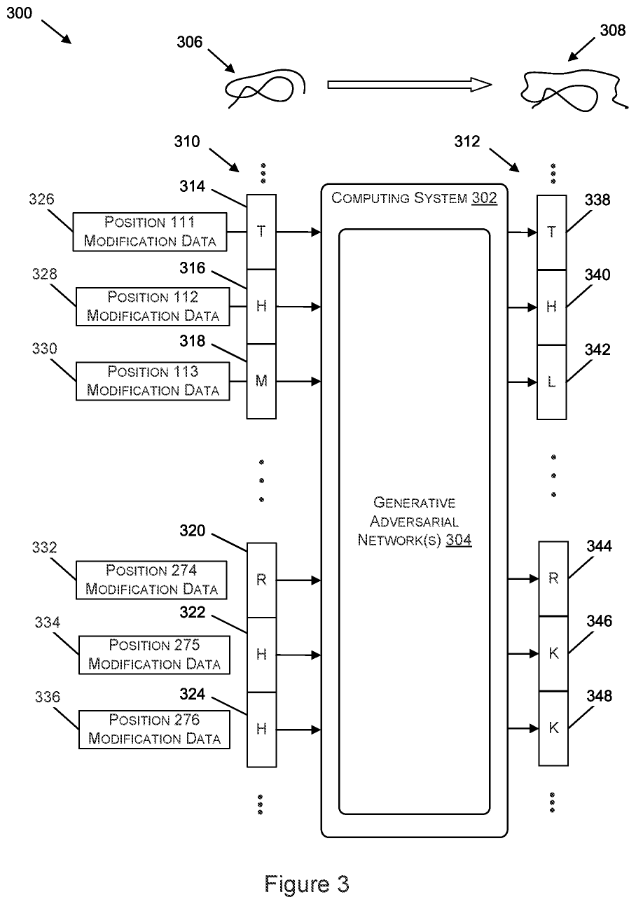

FIG. 3 is a diagram illustrating an example framework 300 to generate target protein sequences using a generative adversarial network based on a template protein sequence and constraint data related to modifications of positions of the template protein sequence, in accordance with some implementations. The framework 300 can include a computing system 302. The computing system 302 can be implemented by one or more computing devices. The one or more computing devices can include one or more server computing devices, one or more desktop computing devices, one or more laptop computing devices, one or more tablet computing devices, one or more mobile computing devices, or combinations thereof. In various implementations, at least a portion of the one or more computing devices can be implemented in a distributed computing environment. For example, at least a portion of the one or more computing devices can be implemented in a cloud computing architecture.

The computing system 302 can include one or more generative adversarial networks 304. The one or more generative adversarial networks 304 can include a conditional generative adversarial network. In various implementations, the one or more generative adversarial networks 304 can include a generating component and a challenging component. The generating component can generate amino acid sequences of proteins and the challenging component can classify the amino acid sequences produced by the generating component as being an amino acid sequence that is included in a set of training or an amino acid sequence that is not included in the set of training data. The set of training data can include amino acid sequences of proteins that have been synthesized and characterized according to one or more analytical tests and/or one or more assays. The output of the challenging component can be based on comparisons between the amino acid sequences produced by the generating component and amino acid sequences included in the set of training data. In illustrative examples, the output of the challenging component can correspond to a probability that an amino acid sequence produced by the generating component is included in the set of training data. As the generating component produces amino acid sequences and as the challenging component produces feedback regarding the amino acid sequences produced by the generating component, the parameters and/or weightings of one or more models implemented by the challenging component and the parameters and/or weightings of one or more models implemented by the generating component can be refined until the one or more models related to the generating component and the one or more models related to the challenging component have been trained and satisfy one or more training criteria. In implementations, the generating component can generate one or more false amino acid sequences of proteins that are not included in the set of training data to try and “trick” the challenging component into classifying the one or more false amino acid sequences of proteins as being included in the set of training data.

The one or more generative adversarial networks 302 can use amino acid sequences of one or more template proteins, such as a template protein 306, and generate one or more amino acid sequences of target proteins, such as a target protein 308. In the illustrative example of FIG. 3, data corresponding to a first amino acid sequence 310 of the template protein 304 can be provided to the computing system 302 and the computing system 302 can generate a second amino acid sequence 312 of the target protein 308. The first amino acid sequence 310 can include a number of amino acids at respective positions, such as amino acid 314 (Threonine) at position 111 of the template protein 306, amino acid 316 (Histidine) at position 112 of the template protein 318, amino acid 318 (Methionine) at position 113 of the template protein 306, amino acid 320 (Arginine) at position 274 of the template protein 306, amino acid 322 (Histidine) at position 275 of the template protein 306, and amino acid 324 (Histidine) at position 276 of the template protein 306. The one or more generative adversarial network 304 can be conditional according to position modification data that corresponds to individual positions of amino acid sequences that are provided to the computing system 302. For example, the amino acids 314, 316, 318, 320, 322, 324 are associated with respective position modification data. To illustrate, the amino acid 314 can be associated with position modification data 326, the amino acid 316 can be associated with position modification data 328, the amino acid 318 can be associated with position modification data 330, the amino acid 320 can be associated with position modification data 332, the amino acid 322 can be associated with position modification data 334, and the amino acid 324 can be associated with position modification data 336.

The position modification data 326, 328, 330, 332, 334, 336 can correspond to constraints on the modification of the individual amino acids 314, 316, 318, 320, 322 324 included in the first sequence of amino acids 310 of the template protein 306. In illustrative examples, the position modification data 326, 328, 330, 332, 334, 336 can indicate penalties that are to be applied by one or more generating components and/or one or more challenging components of the one or more generative adversarial networks 304 in response to modification of the respective individual amino acids 314, 316, 318, 320, 322 324 in the first sequence of amino acids 310. For example, penalties included in the position modification data 326, 328, 330, 332, 334, 336 can be applied to at least one loss function of the one or more generative adversarial networks 304. In additional examples, the position modification data 326, 328, 330, 332, 334, 336 can include probabilities that individual amino acids 314, 316, 318, 320, 322 324 in the first sequence of amino acids 310 can be modified. The position modification data 326, 328, 330, 332, 334, 336 can include numerical values related to probabilities and/or penalties corresponding to the modification of individual amino acids 314, 316, 318, 320, 322 324 included in the first sequence of amino acids 310. To illustrate, the position modification data 326, 328, 330, 332, 334, 336 can include numerical values from 0 to 1, numerical values from −1 to 1, and/or values from 0 to 100. In additional implementations, the position modification data 326, 328, 330, 332, 334, 336 can include one or more functions, such as one or more linear functions or one or more non-linear functions, that include one or more variables are related to the probabilities and/or penalties corresponding to modification of the individual amino acids 314, 316, 318, 320, 322 324 included in first sequence of amino acids 310. In further examples, at least a portion of the position modification data 326, 328, 330, 332, 334, 336 can indicate that the amino acids located at one or more positions 314, 316, 318, 320, 322, 324 are not to be modified by the one or more generative adversarial networks 304. Also, although the illustrative example of FIG. 3 indicates that each position 314, 316, 318, 320, 322, 324 is associated with respective position modification data 326, 328, 330, 332, 334, 336, in additional implementations, at least one of the positions 314, 316, 318, 320, 322, 324 may not be associated with any position modification data. In one or more implementations, position modification data can be associated with one or more groups of positions of the first amino acid sequence.

In various examples, the data corresponding to the first sequence of amino acids 310 of the template protein 306 can be provided to the computing system 302. The first sequence of amino acids 310 and the corresponding position modification data can be used by the one or more generative adversarial networks 304 to generate the second sequence of amino acids 312 that corresponds to the target protein 308. The target protein 308 can be related to, but different from the template protein 306. For example, the one or more generative adversarial networks 304 can modify amino acids at one or more positions of the first sequence of amino acids 310 to produce the second sequence of amino acids 312. To illustrate, the second amino acid sequence 312 includes amino acids 346 and 348 that correspond to amino acids 314, 316 of the first sequence of amino acids 310. That is, amino acid 314 and amino acid 338 are both Threonine and the amino acid 316 and the amino acid 340 are both Histidine. In the illustrative example of FIG. 3, the amino acid 318 and the amino acid 342 are different indicating that the Methionine of amino acid 318 has been changed by the one or more generative adversarial networks 304 to Leucine for amino acid 342. Further, amino acid 320 can correspond to the amino acid 344 with both amino acids 320, 344 being Arginine, while the amino acids 322, 324 in the first amino acid sequence 310 of the template protein 306 have been changed from Histidine to Lysine at amino acids 346, 348 of the second sequence of amino acids 312 of the target protein 308. In addition to modifying amino acids at various positions of the first sequence of amino acids 310 of the template protein 306, the one or more generative adversarial networks 304 can generate the second sequence of amino acids 312 of the target protein 308 by adding amino acids to the first sequence of amino acids 310. The one or more generative adversarial networks 304 can also generate the second sequence of amino acids 312 of the target protein 308 by deleting amino acids from the first sequence of amino acids 310 of the template protein 306.

The target protein 310 can retain one or more characteristics of the template protein 308. The one or more characteristics of the template protein 308 can be maintained in the target protein 310 by maintaining the individual amino acids at various positions of the first amino acid sequence 310 of the template protein 306 in the second amino acid sequence 312 of the target protein 308. The one or more characteristics of the template protein 306 that are also present in the target protein 308 can be preserved by determining one or more positions of the first sequence of amino acids 310 that correspond to the one or more characteristics and minimizing the probability that the one or more generative adversarial networks 304 change the amino acids located at the one or more positions. Additionally, the characteristics of the amino acids in the target protein 308 that are used to replace the initial amino acids in the template protein 306 can be limited. For example, the position modification data for the first sequence of amino acids 310 can indicate that a hydrophobic amino acid is to be replaced by another hydrophobic amino acid. In this way, the target protein 308 can have one or more characteristics of the template protein 306 that are similar or the same. For example, the target protein 308 can have values of one or more biophysical properties that are within a threshold amount of the values of the one or more biophysical properties of the template protein 306. Additionally, the target protein 308 can have functionality that is similar to or the same as functionality of the template protein 306. To illustrate, the target protein 308 and the template protein 306 can both bind to a specified molecule or to a specified type of molecule. In illustrative examples, the template protein 306 can include an antibody that binds to an antigen and the first sequence of amino acids 310 can be modified to the second sequence of amino acids 312 such that the target protein 308 can also bind to the antigen.

In various examples, the position modification data can indicate a penalty and/or a probability associated with changing an amino acid at one position of the template protein 306 to one or more different amino acids in the target protein 308. To illustrate, the position modification data can indicate a first penalty and/or a first probability of changing a threonine of amino acid 314 at position 114 to a serine and a second penalty and/or a second probability of changing the threonine of amino acid 314 at position 114 to a cysteine. The position modification data can, in various implementations, indicate a respective probability and/or a respective penalty for modifying an amino acid at a position of the template protein with respect to each of at least 5 other amino acids, at least 10 other amino acids, at least 15 other amino acids, or at least 20 other amino acids.

The one or more generative adversarial networks 304 can modify template proteins produced by one organism to generate target proteins that correspond to a different organism. For example, the template protein 306 can be produced by mice and the first sequence of amino acids 310 can be modified such that the second sequence of amino acids 312 corresponds to a human protein. In an additional example, the template protein 306 can be produced by humans and the first sequence of amino acids 310 can be modified such that the second sequence of amino acids 312 corresponds to an equine protein. Additionally, the one or more generative adversarial networks 304 can modify template proteins that are produced by one or more genes of a germline to generate proteins that correspond to different germline genes. In illustrative examples, modifications of one or more amino acids of a germline gene of an antibody within a species can have an effect on one or more characteristics of the antibody (e.g., expression level, yield, variable region stability) while maintaining an amount of binding capacity to a specified antigen. Further, in situations where the one or more generative adversarial networks 304 modify amino acid sequences of antibodies, the one or more generative adversarial networks 304 can modify template proteins that correspond to a first antibody isotype, such as an IgE isotype antibody, to generate target antibodies that correspond to a second antibody isotype, such as an IgG isotype antibody.

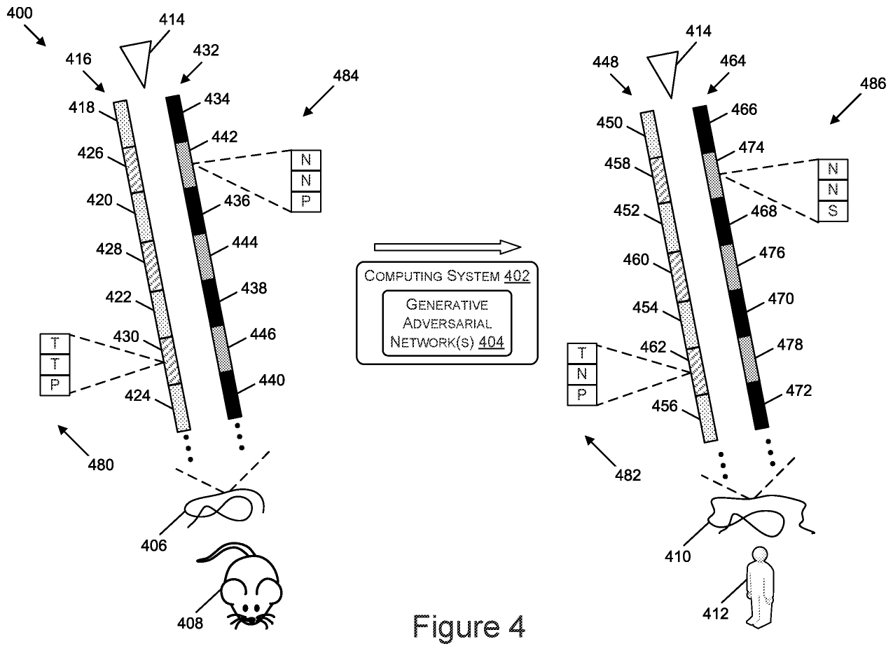

FIG. 4 is a diagram illustrating an example framework 400 to utilize data indicating an antibody sequence of a first organism having specified functionality to generate data corresponding to additional antibody sequences having the specified functionality for a second, different organism, in accordance with some implementations. The framework 400 can include a computing system 402 that can implement one or more generative adversarial networks 404 to modify an amino acid sequence of a template antibody 406 of a first mammal 08 to produce a target antibody 410 of a second mammal 412. In the illustrative example of FIG. 4, the template antibody 406 can be a mouse antibody and the target antibody 410 can correspond to a human antibody. The template antibody 406 can bind to an antigen 414. In addition, the one or more generative adversarial networks 404 can generate the target antibody 410 such that the target antibody 410 has at least a threshold probability of also binding to the antigen 414.

The template antibody 406 can include a first light chain 416. The first light chain 416 can include a variable region having a number of framework regions and a number of hypervariable regions. In various instances, the hypervariable regions can be referred to herein as complementarity determining regions (CDRs). In the illustrative example of FIG. 4, the first light chain 416 can include a first framework region 418, a second framework region 420, a third framework region 422, and a fourth framework region 424. Additionally, the first light chain 416 can include a first CDR 426, a second CDR 428, and a third CDR 430. Although not shown in the illustrative example of FIG. 4, the first light chain 416 can include a constant region that is coupled to the variable region of the first light chain 416 and follows the amino acid sequence of the variable region of the first light chain 416. The constant region of the first light chain 416 and the variable region of the first light chain 416 can form an antigen binding region for the first light chain 416.

The template antibody 406 can also include a first heavy chain 432. The first heavy chain 432 can include a variable region having a number of framework regions and a number of hypervariable regions. The first heavy chain 432 can include a first framework region 434, a second framework region 436, a third framework region 438, and a fourth framework region 440. Further, the first heavy chain 432 can include a first CDR 442, a second CDR 444, and a third CDR 446. Although not shown in the illustrative example of FIG. 4, the first heavy chain 432 can include a number of constant regions that coupled to the variable region of the first heavy chain 432. To illustrate, a first constant region of the first heavy chain 432 can be coupled to the variable region and together the first constant region of the first heavy chain 432 and the variable region of the first heavy chain 432 can form an antigen binding region of the first heavy chain 432. The first heavy chain 432 can also include a crystallizable region that includes two additional constant regions and is coupled to the antigen binding region by a bridge region.

The antigen binding region of the first light chain 416 and the antigen binding region of the first heavy chain 432 can have a shape that corresponds to a shape and a chemical profile of the antigen 414. In various examples, at least a portion of the CDRs 426, 428, 430 of the first light chain 416 and at least a portion of the CDRs 442, 444, 446 of the first heavy chain 432 can include amino acids that interact with amino acids of an epitope region of the antigen 414. In this way, amino acids of at least a portion of the CDRs 426, 428, 430, 442, 444, 446 can interact with amino acids of the antigen 414 through at least one of electrostatic interactions, hydrogen bonds, van der Waals forces, or hydrophobic interactions.

Although not shown in the illustrative example of FIG. 4, the template antibody 406 can also include an additional light chain that is paired with an additional heavy chain. The additional light chain can correspond to the first light chain 416 and the additional heavy chain can correspond to the first heavy chain 432. In illustrative examples, the additional light chain can have a same amino acid sequence as the first light chain 414 and the additional heavy chain can have a same amino acid sequence as the first heavy chain 432. The additional light chain and the additional heavy chain of the template antibody 406 can bind to another antigen molecule that corresponds to the antigen 414.

The one or more generative adversarial networks 404 can generate the target antibody 410 using the amino acid sequences of the regions of the template antibody 406. The target antibody 410 can have one or more portions with amino acid sequences that are different from portions of the amino acid sequence of the template antibody 406. The portions of the amino acid sequence of the template antibody 406 that are changed in relation to the amino acid sequence of the target antibody 410 can be modified such that the target antibody 410 corresponds more closely to an antibody produced by a different species than an antibody produced by a species related to the template antibody 406. In one or more illustrative examples, the one or more generative adversarial networks 404 can modify amino acids included in the variable region of the first light chain 416 and/or amino acids included in the variable region of the first heavy chain 432 to produce the target antibody 410. In various illustrative examples, the one or more generative adversarial networks 404 can modify amino acids included in at least one of one or more of the CDRs 426, 438, 430 of the first light chain 416 or one or more of the CDRs 442, 444, 446 of the first heavy chain 432 to produce the target antibody 410.

The target antibody 410 can include a second light chain 448. The second light chain 448 can correspond to the first light chain 416. In various examples, at least one amino acid of the second light chain 448 can be different from at least one amino acid of the first light chain 416. The second light chain 448 can include a variable region having a number of framework regions and a number of hypervariable regions. The second light chain 448 can include a first framework region 450, a second framework region 452, a third framework region 454, and a fourth framework region 456. Additionally, the second light chain 448 can include a first CDR 458, a second CDR 460, and a third CDR 462. Although not shown in the illustrative example of FIG. 4, the second light chain 448 can include a constant region that is coupled to the variable region of the second light chain 448 and follows the amino acid sequence of the variable region of the second light chain 448. The constant region of the second light chain 448 and the variable region of the second light chain 448 can form an antigen binding region for the second light chain 448.

The target antibody 410 can also include a second heavy chain 464. The second heavy chain 464 can correspond to the first heavy chain 432. In one or more implementations, at least one amino acid of the second heavy chain 464 can be different from at least one amino acid of the first heavy chain 432. The second heavy chain 464 can include a variable region having a number of framework regions and a number of hypervariable regions. The second heavy chain 464 can include a first framework region 466, a second framework region 468, a third framework region 470, and a fourth framework region 472. Further, the second heavy chain 464 can include a first CDR 474, a second CDR 476, and a third CDR 478. Although not shown in the illustrative example of FIG. 4, the second heavy chain 464 can include a number of constant regions that coupled to the variable region of the second heavy chain 464. To illustrate, a first constant region of the second heavy chain 464 can be coupled to the variable region and together the first constant region of the second heavy chain 464 and the variable region of the second heavy chain 464 can form an antigen binding region of the second heavy chain 464. The second heavy chain 464 can also include a crystallizable region that includes two additional constant regions and is coupled to the antigen binding region by a bridge region.

Although the second light chain 448 can have a different amino acid sequence than the first light chain 416 and/or the second heavy chain 464 can have a different amino acid sequence than the first heavy chain 432, the antigen binding region of the second light chain 448 and the antigen binding region of the second heavy chain 464 can have a shape that corresponds to a shape and a chemical profile of the antigen 414. In various examples, at least a portion of the CDRs 458, 460, 462 of the second light chain 448 and at least a portion of the CDRs 474, 476, 478 of the second heavy chain 464 can include amino acids that interact with amino acids of an epitope region of the antigen 414. In this way, amino acids of at least a portion of the CDRs 458, 460, 462, 474, 476, 478 can interact with amino acids of the antigen 414 through at least one of electrostatic interactions, hydrogen bonds, van der Waals forces, or hydrophobic interactions.

Although not shown in the illustrative example of FIG. 4, the target antibody 410 can also include an additional light chain that is paired with an additional heavy chain. The additional light chain can correspond to the second light chain 448 and the additional heavy chain can correspond to the second heavy chain 464. In illustrative examples, the additional light chain can have a same amino acid sequence as the second light chain 448 and the additional heavy chain can have a same amino acid sequence as the second heavy chain 464. The additional light chain and the additional heavy chain of the target antibody 410 can bind to another antigen molecule that corresponds to the antigen 414.

In the illustrative example of FIG. 4, the template antibody 406 can include a first portion having a first amino acid sequence 480 that is different from a second portion of the target antibody 410 that has a second amino acid sequence 482. For example, a threonine molecule included in the first amino acid sequence 480 of the template antibody 406 can be replaced by an asparagine molecule in the second amino acid sequence 482 of a corresponding portion of the target antibody 410. Additionally, the template antibody 406 can include a third portion having a third amino acid sequence 484 that is different from a fourth portion of the target antibody 410 having a fourth amino acid sequence 482. To illustrate, a proline molecule included in the third amino acid sequence 484 of the third portion of the template antibody 406 can be replaced by a serine molecule in the fourth amino acid sequence 486 corresponding to the fourth portion of the target antibody 410.

In various implementations, for each antibody isotype, such as IgA, IgD, IgE, IgG, IgM, the light chain constant regions can be comprised of a same or similar sequence of amino acids and the respective heavy chain constant regions can be comprised of a same or similar sequence of amino acids.

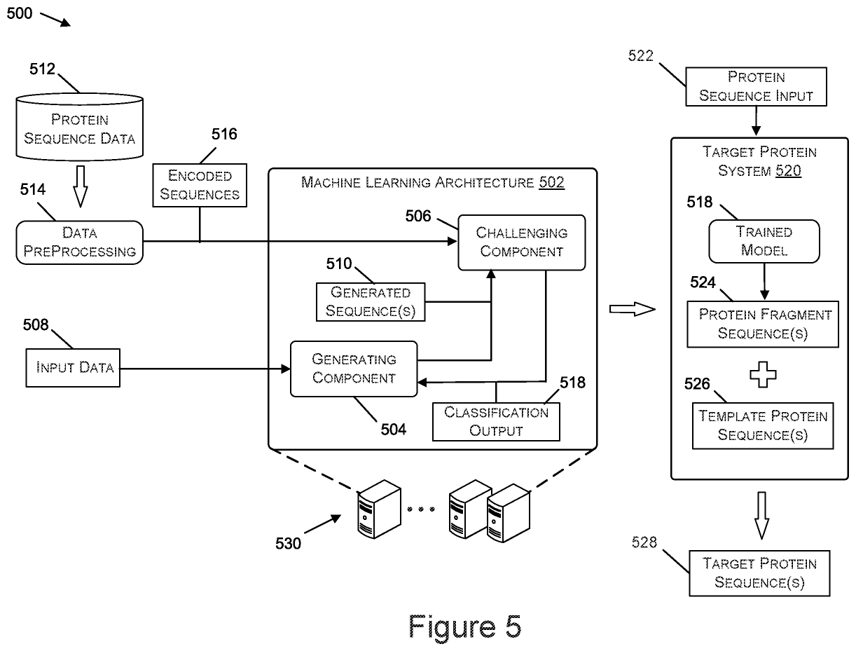

FIG. 5 is a diagram illustrating an example framework 500 to generate target protein sequences using machine learning techniques by combining protein fragment sequences with template protein sequences, in accordance with some implementations. In various examples, the machine learning architecture 502 can generate sequences of fragments of proteins. The sequences of the fragments of proteins can be combined with sequences of protein templates to generate sequences of target proteins. In one or more examples, the machine learning architecture 502 can generate sequences of fragments of antibodies. In these scenarios, the sequences of the antibody fragments can be combined with a template sequence, such as an antibody framework to generate an antibody sequence. In one or more illustrative examples, the machine learning architecture 502 can generate sequences of at least a portion of variable regions of antibodies and the antibody fragment sequences generated by the machine learning architecture 502 can be combined with sequences of additional portions of an antibody to generate a complete antibody sequence. In one or more implementations, the antibody sequence can include one or more light chain variable regions, one or more light chain constant regions, one or more heavy chain variable regions, one or more heavy chain constant regions, or one or more combinations thereof.

The machine learning architecture 502 can include a generating component 504 and a challenging component 506. The generating component 506 can implement one or more models to generate amino acid sequences based on input provided to the generating component 506. In various implementations, the one or more models implemented by the generating component 506 can include one or more functions. The challenging component 506 can generate output indicating whether the amino acid sequences produced by the generating component 504 satisfy various characteristics. The output produced by the challenging component 506 can be provided to the generating component 504 and the one or more models implemented by the generating component 504 can be modified based on the feedback provided by the challenging component 506. The challenging component 506 can compare the amino acid sequences produced by the generating component 504 with amino acid sequences of a library of target proteins and generate an output indicating an amount of correspondence between the amino acid sequences produced by the generating component 504 and the amino acid sequences of target proteins provided to the challenging component 506.

In various implementations, the machine learning architecture 502 can implement one or more neural network technologies. For example, the machine learning architecture 502 can implement one or more recurrent neural networks. Additionally, the machine learning architecture 502 can implement one or more convolution neural networks. In certain implementations, the machine learning architecture 502 can implement a combination of recurrent neural networks and convolutional neural networks. In examples, the machine learning architecture 502 can include a generative adversarial network (GAN). In these situations, the generating component 504 can include a generator and the challenging component 506 can include a discriminator. The challenging component 506 can generate output indicating whether the amino acid sequences produced by the generating component 504 satisfy various characteristics. In various implementations, the challenging component 506 can be a discriminator. In additional situations, such as when the machine learning architecture 502 includes a Wasserstein GAN, the challenging component 506 can include a critic. In additional implementations, the machine learning architecture 502 can include a conditional generative adversarial network (cGAN).

In the illustrative example of FIG. 5, the generating component 504 can obtain input data 508 and the generating component 504 can utilize the input data 508 and one or more models to produce generated sequences 510. The input data 508 can include noise that is produced by a random number generator or noise produced by a pseudo-random number generator. The generated sequences 510 can include amino acid sequences that are represented by a series of letters with each letter indicating an amino acid located at a respective position of a protein. In various examples, the generated sequences 510 can represent fragments of proteins. In one or more illustrative examples, the generated sequences 510 can correspond to fragments of antibodies.

The generated sequence(s) 510 can be analyzed by the challenging component 506 against sequences of proteins included in protein sequence data 512. The protein sequence data 512 can be training data for the machine learning architecture 502. The protein sequence data 512 can be encoded according to a schema. The protein sequence data 512 can include sequences of proteins obtained from one or more data sources that store amino acid sequences of proteins. The one or more data sources can include one or more websites that are searched and information corresponding to amino acid sequences of target proteins is extracted from the one or more websites. Additionally, the one or more data sources can include electronic versions of research documents from which amino acid sequences of target proteins can be extracted. The protein sequence data 512 can be stored in one or more data stores that are accessible to the machine learning architecture 502. The one or more data stores can be connected to the machine learning architecture 502 via a wireless network, a wired network, or combinations thereof. The protein sequence data 512 can be obtained by the machine learning architecture 502 based on requests sent to the data stores to retrieve one or more portions of the protein sequence data 512.

In one or more examples, the protein sequence data 512 can include amino acid sequences of fragments of proteins. For example, the protein sequence data 512 can include sequences of at least one of light chains of antibodies or heavy chains of antibodies. In addition, the protein sequence data 512 can include sequences of at least one of variable regions of antibody light chains, variable regions of antibody heavy chains, constant regions of antibody light chains, constant regions of antibody heavy chains, hinge regions of antibodies, or antigen binding sites of antibodies. In one or more illustrative examples, the protein sequence data 512 can include sequences of complementarity determining regions (CDRs) of antibodies, such as at least one of CDR1, CDR2, or CDR3. In one or more additional illustrative examples, the protein sequence data 512 can include sequences of fragments of T-cell receptors. To illustrate, the protein sequence data 512 can include sequences of antigen binding sites of T-cell receptors, such as one or more CDRs of T-cell receptors.

The amino acid sequences included in the protein sequence data 512 can be subject to data preprocessing 514 before being provided to the challenging component 506. For example, the protein sequence data 512 can be arranged according to a classification system before being provided to the challenging component 506. The data preprocessing 514 can include pairing amino acids included in the target proteins of the protein sequence data 512 with numerical values that can represent structure-based positions within the proteins. The numerical values can include a sequence of numbers having a starting point and an ending point. In an illustrative example, a T can be paired with the number 43 indicating that a Threonine molecule is located at a structure-based position 43 of a specified protein domain type. In illustrative examples, structure-based numbering can be applied to any general protein type, such as fibronectin type III (FNIII) proteins, avimers, antibodies, VHH domains, kinases, zinc fingers, T-cell receptors, and the like.

In various implementations, the classification system implemented by the data preprocessing 516 can include a numbering system that encodes structural position for amino acids located at respective positions of proteins. In this way, proteins having different numbers of amino acids can be aligned according to structural features. For example, the classification system can designate that portions of proteins having particular functions and/or characteristics can have a specified number of positions. In various situations, not all of the positions included in the classification system may be associated with an amino acid because the number of amino acids in a particular region of a protein may vary between proteins. In additional examples, the structure of a protein can be reflected in the classification system. To illustrate, positions of the classification system that are not associated with a respective amino acid can indicate various structural features of a protein, such as a turn or a loop. In an illustrative example, a classification system for antibodies can indicate that heavy chain regions, light chain regions, and hinge regions have a specified number of positions assigned to them and the amino acids of the antibodies can be assigned to the positions according to the classification system. In one or more implementations, the data preprocessing 514 can use Antibody Structural Numbering (ASN) to classify individual amino acids located at respective positions of an antibody.

The output produced by the data preprocessing 514 can include encoded sequences 516. The encoded sequences 516 can include a matrix indicating amino acids associated with various positions of a protein. In examples, the encoded sequences 516 can include a matrix having columns corresponding to different amino acids and rows that correspond to structure-based positions of proteins. For each element in the matrix, a 0 can be used to indicate the absence of an amino acid at the corresponding position and a 1 can be used to indicate the presence of an amino acid at the corresponding position. The matrix can also include an additional column that represents a gap in an amino acid sequence where there is no amino acid at a particular position of the amino acid sequence. Thus, in situations where a position represents a gap in an amino acid sequence, a 1 can be placed in the gap column with respect to the row associated with the position where an amino acid is absent. The generated sequence(s) 510 can also be represented using a vector according to a same or similar number scheme as used for the encoded sequences 516. In some illustrative examples, the encoded sequences 516 and the generated sequence(s) 510 can be encoded using a method that may be referred to as a one-hot encoding method.

In one or more examples, based on similarities and differences between the generated sequence(s) 510 and additional sequences provided to the challenging component 506, such as amino acid sequences included in the protein sequence data 512, the challenging component 506 can generate the classification output 518 to indicate an amount of similarity or an amount of difference between the generated sequence(s) 510 and sequences provided to the challenging component 506 that are included in the protein sequence data 512. In one or more examples, the challenging component 506 can label the generated sequence(s) 510 as zero and the encoded sequences obtained from the protein sequence data 512 as 1. In these situations, the classification output 518 can include a first number from 0 to 1 with respect to one or more amino acid sequences included in the protein sequence data 512.

In one or more additional examples, the challenging component 506 can implement a distance function that produces an output that indicates an amount of distance between the generated sequence(s) 510 and the protein sequences included in the protein sequence data 512. In implementations where the challenging component 506 implements a distance function, the classification output 518 can include a number from −∞ to ∞ indicating a distance between the generated sequence(s) 510 and one or more sequences included in the protein sequence data 512.

The data used to train the machine learning architecture 502 can impact the amino acid sequences produced by the generating component 504. For example, in situations where CDRs of antibodies are included in the protein sequence data 512 provided to the challenging component 506, the amino acid sequences generated by the generating component 504 can correspond to amino acid sequences of antibody CDRs. In another example, in scenarios where the amino acid sequences included in the target protein sequence data 512 provided to the challenging component 506 correspond to CDRs of T-cell receptors, the amino acid sequences produced by the generating component 504 can correspond to sequences of CDRs of T-cell receptors.

After the machine learning architecture 502 has undergone a training process, a trained model 518 can be generated that can produce sequences of proteins. The trained model 518 can include the generating component 504 after a training process has been performed using the protein sequence data 512. In one or more illustrative examples, the trained model 518 include a number of weights and/or a number of parameters of a convolution neural network. The training process for the machine learning architecture 502 can be complete after the function(s) implemented by the generating component 504 and the function(s) implemented by the challenging component 506 converge. The convergence of a function can be based on the movement of values of model parameters toward particular values as protein sequences are generated by the generating component 504 and feedback is obtained from the challenging component 506. In various implementations, the training of the machine learning architecture 502 can be complete when the protein sequences produced by the generating component 504 have particular characteristics. For example, the amino acid sequences generated by the generating component 504 can be analyzed by a software tool that determines at least one of biophysical properties of the amino acid sequences, structural features of the amino acid sequences, or adherence to amino acid sequences corresponding to one or more protein germlines The machine learning architecture 502 can produce the trained model 518 in situations where the amino acid sequences produced by the generating component 504 are determined by the software tool to have one or more specified characteristics. In one or more implementations, the trained model 518 can be included in a target protein system 520 that generates sequences of target proteins.

Protein sequence input 522 can be provided to the trained model 518, and the trained model 518 can produce protein fragment sequences 524. The protein sequence input 522 can include an input vector that can include a random or pseudo-random series of numbers. In one or more illustrative examples, the protein fragment sequences 524 produced by the trained model 518 can be represented as a matrix structure that is the same as or similar to the matrix structure used to represent the encoded sequences 516 and/or the generated sequence(s) 510. In various implementations, the matrices produced by the trained model 518 that comprise the protein fragment sequences 524 can be decoded to produce a string of amino acids that correspond to the sequence of a protein fragment. The protein fragment sequences 524 can include sequences of at least portions of fibronectin type III (FNIII) proteins, avimers, VHH domains, antibodies, kinases, zinc fingers, T-cell receptors, and the like. In one or more illustrative examples, the protein fragment sequences 524 can include sequences of fragments of antibodies. For example, the protein fragment sequences 524 can correspond to portions one or more antibody subtypes, such as immunoglobin A (IgA), immunoglobin D (IgD), immunoglobin E (IgE), immunoglobin G (IgG), or immunoglobin M (IgM). In one or more examples, the protein fragment sequences 524 can include sequences of at least one of one or more antibody light chain variable regions, one or more antibody heavy chain variable regions, one or more antibody light chain constant regions, one or more antibody heavy chain constant regions, or one or more antibody hinge regions. Further, the protein fragment sequences 524 can correspond to additional proteins that bind antigens. In still other examples, the protein fragment sequences 524 can correspond to amino acid sequences that participate in protein-to-protein interactions, such as proteins that have regions that bind to antigens or regions that bind to other molecules.

The target protein system 520 can combine one or more protein fragment sequences 524 with one or more template protein sequences 526 to produce one or more target protein sequences 528. The template protein sequences 526 can include amino acid sequences of portions of proteins that can be combined with the protein fragment sequences 524. For example, a protein fragment sequence 524 can include an amino acid sequence of a variable region of an antibody light chain and a template protein sequence 526 can include an amino acid sequence of a remainder of an antibody. To illustrate, the template protein sequence 526 can include an amino acid sequence that includes a constant region of an antibody light chain. In these scenarios, the target protein sequences 528 can include an amino acid sequence of an antibody light chain. In one or more additional examples, one or more protein fragment sequences 524 can include an amino acid sequence of a variable region of an antibody light chain and an amino acid sequence of a variable region of an antibody heavy chain and one or more template sequences 526 can include amino acid sequences of a constant region of an antibody light chain, a first constant region of an antibody heavy chain, a hinge region of an antibody heavy chain, a second constant region of an antibody heavy chain, and a third constant region of an antibody heavy chain. In these instances, the target protein sequences 528 can include an amino acid sequence of an antibody light chain coupled with an antibody heavy chain.

The target protein system 520 can determine one or more locations of one or more missing amino acids in a template protein sequence 526 and determine one or more amino acids included in one or more protein fragment sequences 524 that can be used to supply the one or more missing amino acid sequences. In various examples, the template protein sequences 526 can indicate locations of missing amino acids within individual template protein sequences 526. In one or more illustrative examples, the trained model 518 can produce protein fragment sequences 524 can correspond to amino acid sequences of antigen binding regions of one or more antibodies. In these scenarios, the target protein system 520 can determine that the template protein sequences 526 are missing at least a portion of the antigen binding regions of one or more antibodies. The target protein system 520 can then extract an amino acid sequence included in the protein fragment sequences 524 that correspond to a missing amino acid sequence of an antigen binding region of a template protein sequence 526. The target protein system 520 can combine the amino acid sequence obtained from the protein fragment sequence 524 with a template protein sequence 526 to generate a target protein sequence 528 that includes the template protein sequence 526 with the antigen binding region supplied by one or more of the protein fragment sequences 524.

Although not shown in the illustrative example of FIG. 5, additional processing can be performed with respect to the target protein sequences 528. For example, the target protein sequences 528 can be evaluated to determine whether the target protein sequences 528 have a specified set of characteristics. To illustrate, one or more metrics can be determined with respect to the target protein sequence(s) 528. For example, metrics that can be determined with respect to the target protein sequences 528 can be related to characteristics of the target protein sequences 528, such as a number of negatively charged amino acids, a number of positively charged amino acids, a number of amino acids interacting to form one or more polar regions, amino acids interacting to form one or more hydrophobic regions, one or more combinations thereof, and the like.

In one or more implementations, the target protein sequences 528 can be subject to sequence filtering. The sequence filtering can parse the target protein sequences 528 to identify one or more of the target protein sequences 528 that correspond to one or more characteristics. For example, the target protein sequences 528 can be analyzed to identify amino acid sequences that have specified amino acids at particular positions. One or more of the target protein sequences 528 can also be filtered to identify amino acid sequences having one or more particular strings or regions of amino acids. In various implementations, the target protein sequences 528 can be filtered to identify amino acid sequences that are associated with a set of biophysical properties based at least partly on similarities between at least one of the target protein sequences 528 and amino acid sequences of additional proteins having the set of biophysical properties.

The machine learning architecture 502 can be implemented by one or more computing devices 530. The one or more computing devices 530 can include one or more server computing devices, one or more desktop computing devices, one or more laptop computing devices, one or more tablet computing devices, one or more mobile computing devices, or combinations thereof. In certain implementations, at least a portion of the one or more computing devices 530 can be implemented in a distributed computing environment. For example, at least a portion of the one or more computing devices 530 can be implemented in a cloud computing architecture. Additionally, although the illustrative example of FIG. 5 shows an implementation of the machine learning architecture 530 that includes a generative adversarial network with a single generating component and a single challenging component, in additional implementations, the machine learning architecture 502 can include multiple generative adversarial networks. Further, each generative adversarial network implemented by the machine learning architecture 502 can include one or more generating components and one or more challenging components. Also, although the illustrative example of FIG. 5 shows the machine learning architecture 502 and the target protein system 520 as separate entities, the machine learning architecture 502 and the target protein system 520 can be implemented as a single system by the one or more computing devices 530.

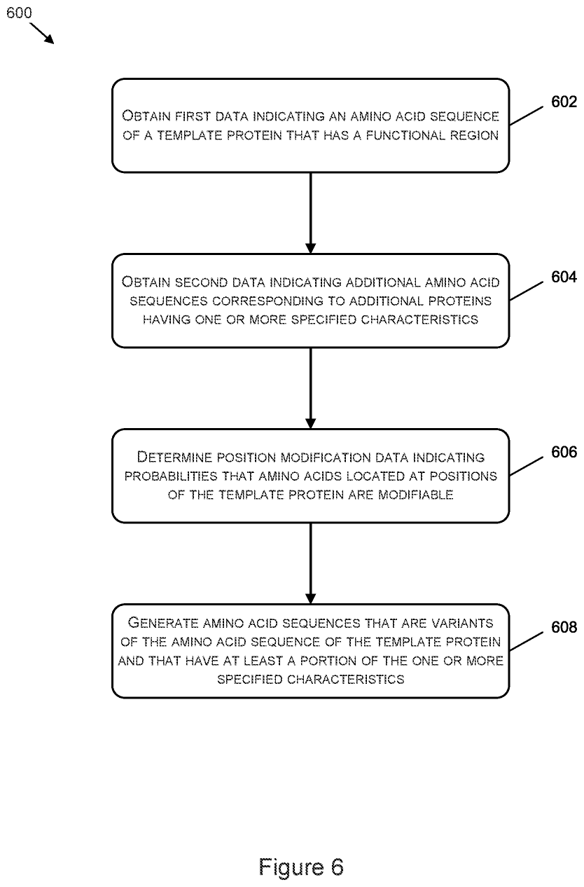

FIG. 6 is a flow diagram illustrating an example method 600 for producing target protein sequences using template protein sequences and position modification data, in accordance with some implementations. The method 600 can include, at operation 602, obtaining first data indicating an amino acid sequence of a template protein that has a functional region. The functional region of the template protein can include amino acids that cause the template protein to bind with another molecule. In various examples, the functional region can have a shape that corresponds to a shape and chemical properties of another molecule. In illustrative examples, the template protein can include an antibody and the functional region can include amino acids that bind to an antigen.

At operation 604, the method 600 can include obtaining second data indicating additional amino acid sequences corresponding to additional proteins having one or more specified characteristics. The one or more specified characteristics can correspond to one or more biophysical properties. The one or more specified characteristics can also correspond to amino acid sequences that can be included in certain types of proteins. For example, the one or more specified characteristics can correspond to amino acid sequences included in human antibodies. To illustrate, the one or more specified characteristics can correspond to amino acid sequences included in framework regions of variable regions of human antibodies. Additionally, the one or more specified characteristics can correspond to amino acid sequences produced by one or more germline genes of human antibodies. The additional proteins can have similarities in relation to the template protein, but the functional region of the template protein may be absent from the additional proteins. For example, the additional proteins can correspond to antibodies, but the antibodies may not bind to the antigen that binds to the functional region of the template protein. In illustrative implementations, the template protein can be produced by a first mammal and the additional proteins can correspond to antibodies produced by a second mammal, such as a human. In these situations, the amino acid sequences included in the second data can include amino acid sequences of human antibodies. In various implementations, the second data can be used as training data for a generative adversarial network.

In addition, at operation 606, the method 600 can include determining position modification data indicating probabilities that amino acids located at positions of the template protein are modifiable. In one or more illustrative examples, the position modification data can indicate that first probabilities to modify amino acids located in a binding region are no greater than about 5% and that second probabilities to modify amino acids located in one or more portions of additional, non-binding regions of a protein are at least 40%. The position modification data can also include penalties for changing amino acids of the amino acid sequence of the template protein. In various examples, the position modification data can be based on a type of amino acid at a position of the amino acid sequence of the template protein. Additionally, the position modification data can be based on a type of amino acid that is replacing an amino acid located at a position of the template protein. For example, the position modification data can indicate a first penalty for modifying amino acids of the template protein having one or more hydrophobic regions and a second penalty that is different than the first penalty for modifying an amino acid of the template protein that is positively charged. Further, the position modification data can indicate a first penalty for modifying an amino acid of the template protein having one or more hydrophobic regions to another amino acid having one or more hydrophobic regions and a second penalty that is different from the first penalty for modifying the amino acid of the template protein having one or more hydrophobic regions to a positively charged amino acid.

Further, at operation 608, the method 600 can include generating amino acid sequences that are variants of the amino acid sequence of the template protein and that have at least a portion of the one or more specified characteristics. The amino acid sequences of the target proteins can be generated using one or more machine learning techniques. In various examples, the amino acid sequences of the variant proteins can be produced using a conditional generative adversarial network.

The amino acid sequences of the variant proteins can have a region that corresponds to the functional region of the template protein, but that have supporting scaffolds or underlying structures, such as one or more framework regions, that are different from that of the template protein. For example, the template protein can be an antibody that binds to an antigen, while the variant proteins can include antibodies having one or more features that are different from features of the template protein that also bind to the antigen, but would not otherwise have a binding region for the antigen without first being modified. In an illustrative example, the template protein can include a human antibody that includes a binding region that binds to an antigen and the additional amino acid sequences can include human antibodies that have one or more biophysical properties that are different from the biophysical properties of the template protein and that do not bind to the antigen. After being trained using the additional amino acid sequences, the amino acid sequence of the template protein, and the position modification data, a generative adversarial network can produce amino acid sequences of variant antibodies that include the binding region of the template protein and that include at least a portion of the biophysical properties of the additional proteins.

In additional illustrative examples, the template protein can correspond to an antibody produced by a mouse that includes a binding region that binds to an antigen. Further, the additional amino acid sequences can correspond to human antibodies that do not bind to the antigen. After being trained using the additional amino acid sequences, the amino acid sequence of the template protein, and the position modification data, a generative adversarial network can produce amino acid sequences of variant antibodies that correspond to human antibodies instead of mouse antibodies and that include the binding region of the template antibody to bind to the antigen. In various examples, the generative adversarial network can modify framework regions of the variable regions of the template mouse antibody to correspond to framework regions of human antibodies. Additionally, the generative adversarial network can produce the variant amino acid sequences of the human antibody such that the amino acid sequence of the binding region of the mouse antibody is present in the variant amino acid sequences and such that the binding region is stable and forms a shape that binds to the antigen.

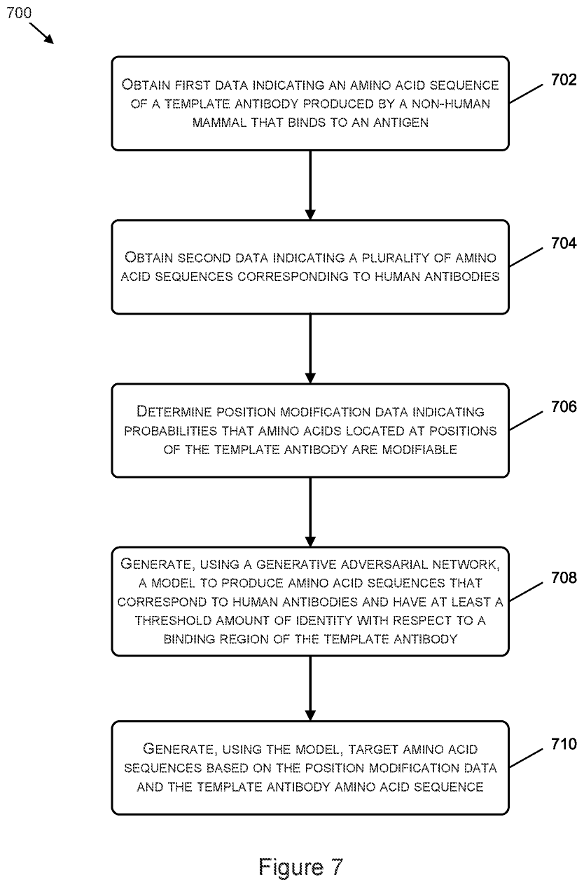

FIG. 7 is a flow diagram illustrating an example method 700 for producing target protein sequences using a generative adversarial network based on template protein sequences, in accordance with some implementations. At 702, the method 700 includes obtaining first data indicating an amino acid sequence of a template antibody produced by a non-human mammal, where the template antibody binds an antigen. The template antibody can include a functional region, such as a CDR, that causes the template antibody to bind to the antigen.

At operation 704, the method 700 includes obtaining second data indicating a plurality of amino acid sequences corresponding to human antibodies. In addition, at operation 706, the method 700 includes determining position modification data indicating probabilities that amino acids located at positions of the template antibody are modifiable. The position modification data can indicate that some positions of the template antibody have relatively high probabilities of being modified and that other positions of the template antibody can have relatively low probabilities of being modified. Positions of the template antibody having relatively high probabilities of being modified can include amino acids at positions that, if modified, are less likely to affect a functional region of the template antibody. Further, the positions of the template antibody having relatively low probabilities of being modified can include amino acids at positions that, if modified, are more likely to affect a functional region of the template antibody. In one or more illustrative examples, the position modification data can indicate that first probabilities to modify amino acids located in an antigen binding region are no greater than about 5% and that second probabilities to modify amino acids located in one or more portions of at least one of the one or more heavy chain framework regions or the one or more light chain framework regions of an antibody are at least 40%. In various examples, the position modification data can indicate penalties that are to be applied by a generative adversarial network to modification of amino acids at positions of the template protein when the generative adversarial network is generating amino acid sequences of target antibodies.

At 708, the method 700 includes generating, using a generative adversarial network, a model to produce amino acid sequences that correspond to human antibodies and that have at least a threshold amount of identity with respect to a binding region of the template antibody. Further, at 710, the method 700 includes generating, using the model, target amino acid sequences based on the position modification data and the template antibody amino acid sequence. In illustrative examples, the amino acid sequences produced by the generative adversarial network can have a scaffolding or underlying structure of human antibodies while having a region that corresponds to the functional region of the template antibody. For example, the amino acid sequences can have constant regions having at least a threshold amount of identity with human antibodies and additional regions, such as CDRs, having a second threshold amount of identity with the functional region of the template antibody.

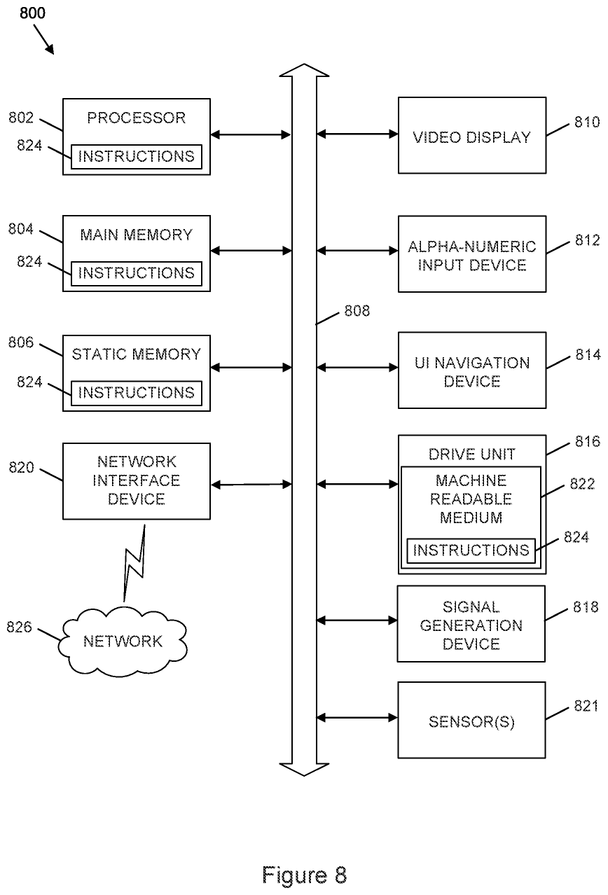

FIG. 8 illustrates a diagrammatic representation of a machine 800 in the form of a computer system within which a set of instructions can be executed for causing the machine 800 to perform any one or more of the methodologies discussed herein, according to an example implementation. Specifically, FIG. 8 shows a diagrammatic representation of the machine 800 in the example form of a computer system, within which instructions (e.g., software, a program, an application, an applet, an app, or other executable code) for causing the machine 800 to perform any one or more of the methodologies discussed herein can be executed. For example, the instructions 824 can cause the machine 800 to implement the frameworks 100, 200, 300, 400, 500 described with respect to FIGS. 1, 2, 3, 4, and 5 respectively, and to execute the methods 600, 700 described with respect to FIGS. 6 and 7, respectively. Additionally, the machine 900 can include or be a part of one or more of the computing devices 144 of FIG. 1 and/or the computing devices 530 of FIG. 5.

The instructions 824 transform the general, non-programmed machine 800 into a particular machine 800 programmed to carry out the described and illustrated functions in the manner described. In additional implementations, the machine 800 operates as a standalone device or may be coupled (e.g., networked) to other machines. In a networked deployment, the machine 800 can operate in the capacity of a server machine or a client machine in a server-client network environment, or as a peer machine in a peer-to-peer (or distributed) network environment. The machine 800 can comprise, but not be limited to, a server computer, a client computer, a personal computer (PC), a tablet computer, a laptop computer, a netbook, a personal digital assistant (PDA), a mobile computing device, a wearable device (e.g., a smart watch), a web appliance, a network router, a network switch, a network bridge, or any machine capable of executing the instructions 824, sequentially or otherwise, that specify actions to be taken by the machine 800. Further, while only a single machine 800 is illustrated, the term “machine” shall also be taken to include a collection of machines 800 that individually or jointly execute the instructions 824 to perform any one or more of the methodologies discussed herein.

Examples of computing device 800 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform operations as described herein. Software can reside (1) on a non-transitory machine readable medium or (2) in a transmission signal. In an example, the software, when executed by the underlying hardware of the circuit, causes the circuit to perform the operations.

A circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In various examples, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., computing device 700) and software architectures that can be deployed in example embodiments.

Example computing device 800 can include a processor 802 (e.g., a central processing unit CPU), a graphics processing unit (GPU) or both), a main memory 804 and a static memory 806, some or all of which can communicate with each other via a bus 808. The computing device 800 can further include a display unit 810, an alphanumeric input device 812 (e.g., a keyboard), and a user interface (UI) navigation device 814 (e.g., a mouse). In an example, the display unit 810, input device 812, and UI navigation device 814 can be a touch screen display. The computing device 800 can additionally include a storage device (e.g., drive unit) 816, a signal generation device 818 (e.g., a speaker), a network interface device 820, and one or more sensors 821, such as a global positioning system (GPS) sensor, compass, accelerometer, or other sensor.

The storage device 816 can include a machine readable medium 822 (also referred to herein as a computer-readable medium) on which is stored one or more sets of data structures or instructions 824 (e.g., software) embodying or utilized by any one or more of the methodologies or functions described herein. The instructions 824 can also reside, completely or at least partially, within the main memory 804, within static memory 806, or within the processor 802 during execution thereof by the computing device 800. In an example, one or any combination of the processor 802, the main memory 804, the static memory 806, or the storage device 816 can constitute machine readable media.

While the machine readable medium 822 is illustrated as a single medium, the term “machine readable medium” can include a single medium or multiple media (e.g., a centralized or distributed database, and/or associated caches and servers) that configured to store the one or more instructions 824. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 824 can further be transmitted or received over a communications network 826 using a transmission medium via the network interface device 820 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### EXAMPLE IMPLEMENTATIONS

Implementation 1. A method comprising: obtaining, by a computing system including one or more computing devices having one or more processors and memory, first data indicating a first amino acid sequence of a template protein, the template protein including a functional region that binds to an additional molecule or that chemically reacts with the additional molecule; obtaining, by the computing system, second data indicating second amino acid sequences corresponding to additional proteins having one or more specified characteristics; obtaining, by the computing system, position modification data indicating, for individual positions of the first amino acid sequence, a probability that an amino acid located at an individual position of the first amino acid sequence is modifiable; generating, by the computing system and using a generative adversarial network, a plurality of third amino acid sequences corresponding to the additional proteins, the plurality of third amino acid sequences being variants of the first amino acid sequence of the template protein, wherein the plurality of third amino acid sequences are generated based on the first data, the second data, and the position modification data.

Implementation 2. The method of implementation 1, wherein individual third amino acid sequences of the plurality of third amino acid sequences include one or more regions having at least a threshold amount of identity with respect to the functional region.

Implementation 3. The method of implementation 1 or 2, wherein the first amino acid sequence includes one or more first groups of amino acids that are produced with respect to a first germline gene and the plurality of third amino acid sequences include one or more second groups of amino acids that are produced with respect to a second germline gene that is different from the first germline gene.

Implementation 4. The method of implementation 3, wherein the one or more second groups of amino acids are included in at least a portion of the second amino acid sequences.

Implementation 5. The method of any one of implementations 1-4, wherein the one or more specified characteristics include values of one or more biophysical properties.

Implementation 6. The method of any one of implementations 1-5, wherein: the template protein is a first antibody; the additional proteins include second antibodies; and the one or more specified characteristics include one or more sequences of amino acids included in one or more framework regions of the second amino acid sequences.

Implementation 7. The method of any one of implementations 1-6, wherein the template protein is produced by a mammal that is not a human and the additional proteins correspond to proteins produced by a human.

Implementation 8. The method of any one of implementations 1-7, comprising: training, by the computing system, a first model using the generative adversarial network and based on the first data, the second data, and the position modification data; obtaining, by the computing system, third data indicating additional amino acid sequences of proteins having a set of biophysical properties; training, by the computing system and using the first model as a generating component of the generative adversarial network, a second model based on the third data; and generating, by the computing system and using the second model; a plurality of fourth amino acid sequences that correspond to proteins that are variants of the template protein and that have at least a threshold probability of having one or more biophysical properties of the set of biophysical properties.

Implementation 9. A method comprising: obtaining, by a computing system including one or more computing devices having one or more processors and memory, first data indicating a first amino acid sequence of an antibody produced by a mammal that is different from a human, the antibody having a binding region that binds to an antigen; obtaining, by the computing system, second data indicating a plurality of second amino acid sequences with individual second amino acid sequences of the plurality of amino acid sequences corresponding to a human antibody; obtaining, by the computing system, position modification data indicating, for individual positions of the first amino acid sequence, a probability that an amino acid located at an individual position of the first amino acid sequence is modifiable; generating, by the computing system and using a generative adversarial network, a model to produce amino acid sequences having at least a first threshold amount of identity with respect to the binding region and at least a second threshold amount of identity with respect to one or more heavy chain framework regions and one or more light chain framework regions of the plurality of second amino acid sequences; and generating, by the computing system and using the model, a plurality of third amino acid sequences based on the position modification data and the first amino acid sequence.

Implementation 10. The method of implementation 9, wherein the position modification data indicates that first probabilities to modify amino acids located in the binding region are no greater than about 5% and that second probabilities to modify amino acids located in one or more portions of at least one of the one or more heavy chain framework regions or the one or more light chain framework regions of the antibody are at least 40%.

Implementation 11. The method of implementation 9 or 10, wherein the position modification data indicates penalties to apply to modification of amino acids of the antibody with respect to generating the plurality of third amino acid sequences.

Implementation 12. The method of implementation 11, wherein the position modification data indicates that an amino acid located at a first position of the first amino acid sequence of the antibody has a first penalty for being changed to a first type of amino acid and a second penalty for being changed to a second type of amino acid.

Implementation 13. The method of implementation 12, wherein the amino acid has one or more hydrophobic regions, the first type of amino acid corresponds to hydrophobic amino acids, and the second type of amino acid corresponds to positively charged amino acids.

Implementation 14. A system comprising: one or more hardware processors; one or more non-transitory computer-readable storage media storing instructions that, when executed by the one or more hardware processors, cause the one or more hardware processors to perform operations comprising: obtaining first data indicating a first amino acid sequence of a template protein, the template protein including a functional region that binds to an additional molecule or that chemically reacts with the additional molecule; obtaining second data indicating second amino acid sequences corresponding to additional proteins having one or more specified characteristics; obtaining position modification data indicating, for individual positions of the first amino acid sequence, a probability that an amino acid located at an individual position of the first amino acid sequence is modifiable; generating, using a generative adversarial network, a plurality of third amino acid sequences corresponding to the additional proteins, the plurality of third amino acid sequences being variants of the first amino acid sequence of the template protein, wherein the plurality of third amino acid sequences are generated based on the first data, the second data, and the position modification data.

Implementation 15. The system of implementation 14, wherein individual third amino acid sequences of the plurality of third amino acid sequences include one or more regions having at least a threshold amount of identity with respect to the functional region.

Implementation 16. The system of implementation 14 or 15, wherein the first amino acid sequence includes one or more first groups of amino acids that are produced with respect to a first germline gene and the plurality of third amino acid sequences include one or more second groups of amino acids that are produced with respect to a second germline gene that is different from the first germline gene.

Implementation 17. The system of implementation 16, wherein the one or more second groups of amino acids are included in at least a portion of the second amino acid sequences.

Implementation 18. The system of any one of implementations 14-17, wherein the one or more specified characteristics include values of one or more biophysical properties.

Implementation 19. The system of any one of implementations 14-18, wherein: the template protein is a first antibody; the additional proteins include second antibodies; and the one or more specified characteristics include one or more sequences of amino acids included in one or more framework regions of the second amino acid sequences.

Implementation 20. The system of any one of implementations 14-19, wherein the template protein is produced by a mammal that is not a human and the additional proteins correspond to proteins produced by a human.

Implementation 21. The system of any one of implementations 14-20, wherein the one or more non-transitory computer-readable storage media storing additional instructions that, when executed by the one or more hardware processors, cause the one or more hardware processors to perform additional operations comprising: training a first model using the generative adversarial network and based on the first data, the second data, and the position modification data; obtaining third data indicating additional amino acid sequences of proteins having a set of biophysical properties; training, using the first model as a generating component of the generative adversarial network, a second model based on the third data; and generating, using the second model, a plurality of fourth amino acid sequences that correspond to proteins that are variants of the template protein and that have at least a threshold probability of having one or more biophysical properties of the set of biophysical properties.

Implementation 22. A system comprising: one or more hardware processors; one or more non-transitory computer-readable storage media storing instructions that, when executed by the one or more hardware processors, cause the one or more hardware processors to perform operations comprising: obtaining first data indicating a first amino acid sequence of an antibody produced by a mammal that is different from a human, the antibody having a binding region that binds to an antigen; obtaining second data indicating a plurality of second amino acid sequences with individual second amino acid sequences of the plurality of amino acid sequences corresponding to a human antibody; obtaining position modification data indicating, for individual positions of the first amino acid sequence, a probability that an amino acid located at an individual position of the first amino acid sequence is modifiable; generating, using a generative adversarial network, a model to produce amino acid sequences having at least a first threshold amount of identity with respect to the binding region and at least a second threshold amount of identity with respect to one or more heavy chain framework regions and one or more light chain framework regions of the plurality of second amino acid sequences; and generating, using the model, a plurality of third amino acid sequences based on the position modification data and the first amino acid sequence.

Implementation 23. The system of implementation 22, wherein the position modification data indicates that first probabilities to modify amino acids located in the binding region are no greater than about 5% and that second probabilities to modify amino acids located in one or more portions of at least one of the one or more heavy chain framework regions or the one or more light chain framework regions of the antibody are at least 40%.

Implementation 24. The system of implementation 22 or 23, wherein the position modification data indicates penalties to apply to modification of amino acids of the antibody with respect to generating the plurality of third amino acid sequences.

Implementation 25. The system of implementation 24, wherein the position modification data indicates that an amino acid located at a first position of the first amino acid sequence of the antibody has a first penalty for being changed to a first type of amino acid and a second penalty for being changed to a second type of amino acid.

Implementation 26. The system of implementation 25, wherein the amino acid has one or more hydrophobic regions, the first type of amino acid corresponds to hydrophobic amino acids, and the second type of amino acid corresponds to positively charged amino acids.

1. A system comprising:
one or more hardware processors;
one or more non-transitory computer-readable storage media storing instructions that, when executed by the one or more hardware processors, cause the one or more hardware processors to perform operations comprising:
obtaining first data indicating a first amino acid sequence of an antibody produced by a mammal that is different from a human, the antibody having a binding region that binds to an antigen;
obtaining second data indicating a plurality of second amino acid sequences with individual second amino acid sequences of the plurality of amino acid sequences corresponding to a human antibody;
determining position modification data indicating, for individual positions of the first amino acid sequence, a probability that an amino acid located at an individual position of the first amino acid sequence is modifiable;
generating, using a generative adversarial network, a model to produce amino acid sequences having at least a first threshold amount of identity with respect to the binding region and at least a second threshold amount of identity with respect to one or more heavy chain framework regions and one or more light chain framework regions of the plurality of second amino acid sequences; and
generating, using the model, a plurality of third amino acid sequences based on the position modification data and the first amino acid sequence.

2. The system of claim 1, wherein the position modification data indicates that first probabilities to modify amino acids located in the binding region are no greater than about 5% and that second probabilities to modify amino acids located in one or more portions of at least one of the one or more heavy chain framework regions or the one or more light chain framework regions of the antibody are at least 40%.

3. The system of claim 1, wherein the position modification data indicates penalties to apply to modification of amino acids of the antibody with respect to generating the plurality of third amino acid sequences.

4. The system of claim 3, wherein the position modification data indicates that an amino acid located at a first position of the first amino acid sequence of the antibody has a first penalty for being changed to a first type of amino acid and a second penalty for being changed to a second type of amino acid.

5. The system of claim 4, wherein the amino acid has one or more hydrophobic regions, the first type of amino acid corresponds to hydrophobic amino acids, and the second type of amino acid corresponds to positively charged amino acids.

6. The system of claim 1, wherein the one or more non-transitory computer-readable storage media store additional instructions that, when executed by the one or more hardware processors, cause the one or more hardware processors to perform additional operations comprising:
performing a training process to produce the model that includes:
producing, by a generating component of the generative adversarial network, first amino acid sequences using amino acid sequences of template proteins and the position modification data;
analyzing, by a challenging component of the generative adversarial network, the first amino acid sequences with respect to amino acid sequences of target proteins to determine classification output that is provided to the generating component, the classification input indicating amounts of differences between respective first amino acid sequences and respective second amino acid sequences; and
determining at least one of parameters or coefficients of the model based on the amount of differences between the respective first amino acid sequences and the respective second amino acid sequences being minimized

7. The system of claim 6, wherein the one or more non-transitory computer-readable storage media storing additional instructions that, when executed by the one or more hardware processors, cause the one or more hardware processors to perform additional operations comprising:
obtaining additional data indicating additional amino acid sequences of proteins having a set of biophysical properties;
performing an additional training process of an additional model, using the model as an additional generating component of the generative adversarial network, that includes:
producing, by the additional generating component, third amino acid sequences using input data;
analyzing, by an additional challenging component of the generative adversarial network, the third amino acid sequences with respect to the additional amino acid sequences to determine additional classification output that is provided to the additional generating component, the additional classification input indicating amounts of differences between respective third amino acid sequences and respective additional amino acid sequences; and
determining at least one of parameters or coefficients of the additional model based on the amount of differences between the respective third amino acid sequences and the respective additional amino acid sequences being minimized

8. A method comprising:
obtaining, by a computing system including one or more computing devices having one or more processors and memory, first data indicating a first amino acid sequence of a template protein, the template protein including a functional region that binds to an additional molecule or that chemically reacts with the additional molecule;
obtaining, by the computing system, second data indicating second amino acid sequences corresponding to additional proteins having one or more specified characteristics;
determining, by the computing system, position modification data indicating, for individual positions of the first amino acid sequence, a probability that an amino acid located at an individual position of the first amino acid sequence is modifiable; and
generating, by the computing system and using a generative adversarial network, a plurality of third amino acid sequences corresponding to the additional proteins, the plurality of third amino acid sequences being variants of the first amino acid sequence of the template protein, wherein the plurality of third amino acid sequences are generated based on the first data, the second data, and the position modification data.

9. The method of claim 8, wherein individual third amino acid sequences of the plurality of third amino acid sequences include one or more regions having at least a threshold amount of identity with respect to the functional region.

10. The method of claim 8, wherein the first amino acid sequence includes one or more first groups of amino acids that are produced with respect to a first germline gene and the plurality of third amino acid sequences include one or more second groups of amino acids that are produced with respect to a second germline gene that is different from the first germline gene.

11. The method of claim 10, wherein the one or more second groups of amino acids are included in at least a portion of the second amino acid sequences.

12. The method of claim 8, wherein the one or more specified characteristics include values of one or more biophysical properties.

13. The method of claim 8, wherein:
the template protein is a first antibody;
the additional proteins include second antibodies; and
the one or more specified characteristics include one or more sequences of amino acids included in one or more framework regions of the second amino acid sequences.

14. The method of claim 8, wherein the template protein is produced by a mammal that is not a human and the additional proteins correspond to proteins produced by a human.

15. The method of claim 8, comprising:
training, by the computing system, a first model using the generative adversarial network and based on the first data, the second data, and the position modification data;
obtaining, by the computing system, third data indicating additional amino acid sequences of proteins having a set of biophysical properties;
training, by the computing system and using the first model as a generating component of the generative adversarial network, a second model based on the third data; and
generating, by the computing system and using the second model; a plurality of fourth amino acid sequences that correspond to proteins that are variants of the template protein and that have at least a threshold probability of having one or more biophysical properties of the set of biophysical properties.

16. A method comprising:
obtaining, by a computing system including one or more computing devices having one or more processors and memory, first data indicating a first amino acid sequence of an antibody produced by a mammal that is different from a human, the antibody having a binding region that binds to an antigen;
obtaining, by the computing system, second data indicating a plurality of second amino acid sequences with individual second amino acid sequences of the plurality of amino acid sequences corresponding to a human antibody;
determining, by the computing system, position modification data indicating, for individual positions of the first amino acid sequence, a probability that an amino acid located at an individual position of the first amino acid sequence is modifiable;
generating, by the computing system and using a generative adversarial network, a model to produce amino acid sequences having at least a first threshold amount of identity with respect to the binding region and at least a second threshold amount of identity with respect to one or more heavy chain framework regions and one or more light chain framework regions of the plurality of second amino acid sequences; and
generating, by the computing system and using the model, a plurality of third amino acid sequences based on the position modification data and the first amino acid sequence.

17. The method of claim 16, wherein the position modification data indicates that first probabilities to modify amino acids located in the binding region are no greater than about 5% and that second probabilities to modify amino acids located in one or more portions of at least one of the one or more heavy chain framework regions or the one or more light chain framework regions of the antibody are at least 40%.

18. The method of claim 16, wherein the position modification data indicates penalties to apply to modification of amino acids of the antibody with respect to generating the plurality of third amino acid sequences.

19. The method of claim 18, wherein the position modification data indicates that an amino acid located at a first position of the first amino acid sequence of the antibody has a first penalty for being changed to a first type of amino acid and a second penalty for being changed to a second type of amino acid.

20. The method of claim 19, wherein the amino acid has one or more hydrophobic regions, the first type of amino acid corresponds to hydrophobic amino acids, and the second type of amino acid corresponds to positively charged amino acids.
